(19) United States
(12) Patent Application Publication
(10) Pub. No.: US 2026/0013861 A1
(43) Pub. Date: Jan. 15, 2026

(54) EPINEURIAL COAPTATION IMPLANTS, INSTRUMENTS AND METHODS OF USE

(71) Applicant: Epineurial Coaptation Technology, LLC, Cheshire, CT (US)

(72) Inventors: John PEPPER, Cheshire, CT (US); Steven S. SMITH, Glastonbury, CT (US)

(73) Assignee: Epineurial Coaptation Technology, LLC, Cheshire, CT (US)

(21) Appl. No.: 19/337,475

(22) Filed: Sep. 23, 2025

Related U.S. Application Data
(63) Continuation of application No. PCT/US2024/020669, filed on Mar. 20, 2024.
(60) Provisional application No. 63/491,868, filed on Mar. 23, 2023.

(51) Int. Cl. A61B 17/11; A61B 17/30; A61B 17/32; A61B 17/3205; A61B 90/00

(52) U.S. Cl. CPC A61B 17/1128; A61B 17/3205; A61B 2017/306; A61B 2017/320052; A61B 2090/061

(57) ABSTRACT

A conduit carrier includes a carrier housing including first and second contact ends and a vacuum channel extending therebetween. The vacuum channel is configured to connect to a vacuum source through the first contact end. An engagement mechanism is connected to the second contact end. The engagement mechanism is configured to releasably engage a conduit during a nerve repair procedure. A separator is configured to extend from the second contact end. When the engagement mechanism is engaged with the conduit during the nerve repair procedure: the vacuum channel is connected through the second contact end to a vacuum port of the conduit to enable the vacuum source to pull a vacuum on an interior of the conduit, and the separator extends into the interior of the conduit through the vacuum port to provide a predetermined regeneration gap between a first and a second nerve stump inserted into the conduit.

### CROSS REFERENCE TO RELATED APPLICATIONS

This application is a continuation of International Patent Application No. PCT/US2024/020669, filed on Mar. 20, 2024, and entitled “Epineurial Coaptation Implants, Instruments and Methods,” which claims benefit of priority of U.S. provisional application 63/491,868, filed Mar. 23, 2023, entitled “Epineurial Coaptation Implants, Instruments and Methods of Use,” the disclosures of these applications are hereby incorporated herein by reference in their entirety.

### TECHNICAL FIELD

This invention is in the field of repair of damaged peripheral nerves.

### BACKGROUND OF THE INVENTION

Nerve damage is caused by laceration, crushing, tearing or puncture. The patient will experience loss of sensory ability and motor ability. The hand and wrist are the sites of most nerve repairs, though they are performed in all appendages.

Nerves consist of many individual nerve cells, axons, grouped together in a funiculus, wrapped in perineurium, which are grouped with other grouped axons, and blood vessels, all contained in an outer sheath called the epineurium. The epineurium is thin, flexible, and nearly translucent. When a nerve is cut, a scar will form over the end.

The nerve cells, axons, are capable of elongating from proximal stump distally, in a growth cone, at a rate of 1 millimeter per day. These cannot penetrate a scar, and its chemistry inhibits axon growth. Many times, the scar must be trimmed off surgically.

Repair depends on distance between viable ends of the stumps. If there is no gap, the stumps can be sutured directly to each other.

The alignment and drawing together of stumps to each other is defined as coaptation in medical terms.

In injuries with gaps under 5 millimeters, if the stumps are approximated, the cells will connect with the distal stump and regain of function is possible. The use of conduits has been an effective way to coapt the stumps. Larger gaps are treated with resorbable scaffolds or grafts. (Griffin J W. Peripheral nerve repair and reconstruction. J Bone Joint Surg Am. 2013 Dec. 4;95(23):2144-51)

Effective repair of injured nerves as long been a tedious problem of repair and remains largely the purview of micro surgically trained plastic or hand surgeons. The accurate, end-to-end anastomosis of the nerves to create a stable, secure connection without causing overlapping of the fascicles remains a challenging surgical problem even for skilled and experienced surgeons.

The handling of conduits and grafts is typically done manually. Coaptation is done with manually placed fine sutures inside the scaffold to the stump, then suture ends are pulled to move stump ends into the scaffold tube. The nerve attachment is done via passing an 8-0 suture through the epineural tissue, which is thin and weak. Tear out of suture is common, requiring time consuming repetition of those steps. Sometime passing needles through conduits breaks the needles, due to their small size.

To coapt the nerves into the conduit with sutures typically means the conduit has to be over sized, and the control of advancing axons is lessened, due to the residual large radial gap.

Stump sizing for selecting the proper sized conduit is typically done with a basic linear ruler. Rulers are often 1 cm wide, due in part to the sizing information being adjacent to the length increments, and are comparatively very large when the repair is being done in the hand of a small female patient. Rulers rely on the surgeon to align the ruler by visually putting the zero on one side, and looking at a visual alignment with the other side of the nerve and a mark on the scale. There are two opportunities for a parallax error, and an inaccurate measurement.

The pursuit of a secure, simple and effective method repair of these nerve ends has led to the development of this device.

### SUMMARY OF THE INVENTION

The present invention provides a vacuum reducing instrument that will allow coaptation of both stumps of a cut nerve, without collapsing the conduit, having a simple release, that requires minimal dissection, with associated instruments facilitating simple nerve stump preparation, quick and accurate nerve size measurement, and ease of suturing tools. The disclosed invention will greatly simplify the process of repair making quality, effective nerve repairs more efficient and more ideal for subsequent nerve regrowth. Additionally, the device will allow for markedly increased control of the nerve ends during the anastomotic process allowing for greater end-to-end gap control and gentle tissue handling. These advances will make quality nerve repair much more fluid and quick for surgeons and even make nerve repair available to surgeons without extensive microsurgical training and experience.

A conduit in accordance with one or more aspects of the present disclosure includes a conduit body including opposing first and second open end portions and a hollow conduit interior extending therebetween. The conduit interior is configured to receive a first nerve stump through the first open end portion and a second nerve stump through the second open end portion during a nerve repair procedure on a patient. A vacuum port extends through an outer wall of the conduit body. The vacuum port is configured to connect to a vacuum source associated with a conduit carrier to pull a vacuum on the conduit interior while inserting the nerve stumps into the first and second open end portions. At least one radial support structure is positioned on the outer wall of the conduit body. The at least one radial support structure is configured to structurally support the conduit body when the vacuum is being applied.

A conduit carrier in accordance with one or more aspects of the present disclosure includes a carrier housing including first and second contact ends and a vacuum channel extending therebetween. The vacuum channel is configured to connect to a vacuum source through the first contact end. An engagement mechanism is connected to the second contact end. The engagement mechanism is configured to releasably engage a conduit during a nerve repair procedure on a patient. A separator is configured to extend from the second contact end. When the engagement mechanism is engaged with the conduit during the nerve repair procedure, the vacuum channel is connected through the second contact end to a vacuum port of the conduit to enable the vacuum source to pull a vacuum on an interior of the conduit, and the separator extends into the interior of the conduit through the vacuum port to provide a predetermined regeneration gap between a first and a second nerve stump inserted into the conduit.

A nerve sizing instrument for sizing a nerve stump during a nerve repair procedure on a patient in accordance with one or more aspects of the present disclosure includes the nerve sizing instrument including a plurality of U-shaped recesses. Respective recesses of the plurality of U-shaped recesses are configured to receive therein a nerve stump having up to a predetermined nerve stump maximum diameter that is different from any other nerve stump maximum diameter associated with any other respective recess.

A scar trimmer for trimming a nerve stump during a nerve repair procedure on a patient in accordance with one or more aspects of the present disclosure includes a longitudinally extending housing assembly having a forward end and a rearward end. The forward end has a forward boss disposed thereon. The forward boss has a stump cutting cavity disposed therein. The stump cutting cavity is configured to receive a nerve stump extended therethrough. An internal slider is disposed within an interior of the housing assembly. The internal slider is movable between a forward position and a rearward position relative to the housing assembly. A blade is slidably captured by the internal slider. When a nerve stump is extended through the stump cutting cavity and the internal slider is moved between the rearward position and the forward position, the blade is slid through the stump cutting cavity to trim a scarred end of the nerve stump off.

A method of performing a nerve repair procedure on a patient in accordance with one or more aspects of the present disclosure includes fitting a first nerve stump into a U-shaped recess of a nerve sizing instrument to determine a diameter of the first nerve stump. A conduit and a conduit carrier are selected based on the determined diameter of the first nerve stump. The selected conduit is releasably engaged with the selected conduit carrier. A first separator of the conduit carrier is extended into a vacuum port of the conduit. A vacuum channel of the conduit carrier is connected to the vacuum port of the conduit. A vacuum is pulled, via the vacuum channel, on an interior of the conduit. The first and the second nerve stumps are inserted into first and second open ends respectively of the conduit while pulling the vacuum. The first separator is utilized to provide a predetermined regeneration gap between the first and second nerve stumps.

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits and advantages described herein.

### BRIEF DESCRIPTION OF THE DRAWINGS

The accompanying drawings, which are incorporated in and constitute a part of the specification, illustrate embodiments of the disclosure and together with the detailed description herein, serve to explain the principles of the disclosure. It is emphasized that, in accordance with the standard practice in the industry, various features are not drawn to scale. In fact, the dimensions of the various features may be arbitrarily increased or reduced for clarity of discussion. The drawings are only for purposes of illustrating preferred embodiments and are not to be construed as limiting the disclosure.

FIG. 1 is a side view of a prior art conduit;

FIG. 2 is a sectional side view of a prior art conduit carrier and another prior art conduit mounted therein;

FIG. 3 is a sectional top view of the prior art conduit and conduit carrier of FIG. 2;

FIG. 4 is a cross-sectional view of the prior art conduit carrier of FIG. 3 taken along the line 4-4 of FIG. 3;

FIG. 5 is a perspective view of the conduit with tabs, and vacuum port flaps open, in accordance with an aspect of the present disclosure;

FIG. 6 is a perspective view of the conduit with tabs and the vacuum port flaps closed, in accordance with an aspect of the present disclosure;

FIG. 7 is a side view of the conduit with tabs and simple vacuum port, in accordance with an aspect of the present disclosure;

FIG. 8 is an end view of the conduit with fixed tabs, in accordance with an aspect of the present disclosure;

FIG. 9 is a perspective view of the conduit with the mobile tabs and vacuum port flaps open, in accordance with an aspect of the present disclosure;

FIG. 10 is a perspective view of the conduit with the mobile tabs and vacuum port flaps closed, in accordance with an aspect of the present disclosure;

FIG. 11 is a perspective view of the conduit with the mobile tabs and a simple vacuum port, in accordance with an aspect of the present disclosure;

FIG. 12 is a perspective view of the conduit with rolled back ends and unrolling tabs, in accordance with an aspect of the present disclosure;

FIG. 13 is a perspective view of the less stiff conduit with rolled back ends and unrolling tabs, in accordance with an aspect of the present disclosure;

FIG. 14 is a perspective view of the less stiff conduit with rolled back ends and unrolling tabs partially deployed, in accordance with an aspect of the present disclosure;

FIG. 15 is a perspective view of the less stiff conduit with unrolled ends and unrolling tabs fully extended, in accordance with an aspect of the present disclosure;

FIG. 16 is a perspective view of the less stiff conduit with unrolling tabs removed, in accordance with an aspect of the present disclosure;

FIG. 17 is a perspective view of the sizing instrument, in accordance with an aspect of the present disclosure;

FIG. 18 is a side view of the sizing instrument, in accordance with an aspect of the present disclosure;

FIG. 19 is a bottom view of the sizing instrument, in accordance with an aspect of the present disclosure;

FIG. 20 is a perspective view of the sizing instrument with a nerve stump, in accordance with an aspect of the present disclosure;

FIG. 21 is a side view of the sizing instrument with a nerve stump, in accordance with an aspect of the present disclosure;

FIG. 22 is a close up of the stump in a sizing trough, in accordance with an aspect of the present disclosure;

FIG. 23 is a perspective view of the scar trimmer, in accordance with an aspect of the present disclosure;

FIG. 24 is a side view of the scar trimmer with the blade back, in accordance with an aspect of the present disclosure;

FIG. 25 is a side view of the scar trimmer with the blade forward, in accordance with an aspect of the present disclosure;

FIG. 26 is a side view of the scar trimmer with a large opening, in accordance with an aspect of the present disclosure;

FIG. 27 is a top view of the scar trimmer, in accordance with an aspect of the present disclosure;

FIG. 28 is a sectional view of the scar trimmer with a large opening and oblique edge blade, in accordance with an aspect of the present disclosure;

FIG. 29 is an exploded perspective top view of the scar trimmer, in accordance with an aspect of the present disclosure;

FIG. 30 is a side view of the scar trimmer with an open slot, with blade retracted, in accordance with an aspect of the present disclosure;

FIG. 31 is a perspective view of the scar trimmer and a scarred stump, in accordance with an aspect of the present disclosure;

FIG. 32 is a perspective view of a nerve with clean cut end, in accordance with an aspect of the present disclosure;

FIG. 33 is a perspective view of the conduit carrier, in accordance with an aspect of the present disclosure;

FIG. 34 is a side view of the conduit carrier with the jaws closed, in accordance with an aspect of the present disclosure;

FIG. 35 is a side view of the conduit carrier with the jaws open, in accordance with an aspect of the present disclosure;

FIG. 36 is a side view of the conduit carrier with the jaws open and conduit disengaged, in accordance with an aspect of the present disclosure;

FIG. 37 is an exploded perspective view of the conduit carrier, in accordance with an aspect of the present disclosure;

FIG. 38 is a side view of the conduit carrier with the jaws closed, in accordance with an aspect of the present disclosure;

FIG. 39 is a top view of the conduit carrier with the jaws closed, in accordance with an aspect of the present disclosure;

FIG. 40 is an enlarged sectional view of the conduit carrier with the jaws closed, in accordance with an aspect of the present disclosure;

FIG. 41 is an enlarged side view of the conduit carrier showing the separator, in accordance with an aspect of the present disclosure;

FIG. 42 is a side view of the conduit carrier with the jaws open, in accordance with an aspect of the present disclosure;

FIG. 43 is a top view of the conduit carrier with the jaws open, in accordance with an aspect of the present disclosure;

FIG. 44 is an enlarged sectional view of the conduit carrier with the jaws open, in accordance with an aspect of the present disclosure;

FIG. 45 is an enlarged sectional view of the conduit carrier, in accordance with an aspect of the present disclosure;

FIG. 46 is a perspective view of the conduit carrier central housing for mobile separator, in accordance with an aspect of the present disclosure;

FIG. 47 is a perspective view of the conduit carrier central housing with a fixed separator, in accordance with an aspect of the present disclosure;

FIG. 48 is a side view of the conduit carrier engaged with two nerve stumps, in accordance with an aspect of the present disclosure;

FIG. 49 is a top view of the conduit carrier engaged with two nerve stumps, in accordance with an aspect of the present disclosure;

FIG. 50 is a sectional view of the conduit carrier engaged with two nerve stumps, in accordance with an aspect of the present disclosure;

FIG. 51 is a sectional view of the carriers closed jaws pinching the conduit's tabs, in accordance with an aspect of the present disclosure;

FIG. 52 is a side view of the jaw, in accordance with an aspect of the present disclosure;

FIG. 53 is a bottom view of the jaw, in accordance with an aspect of the present disclosure;

FIG. 54 is a sectional view of the jaw, in accordance with an aspect of the present disclosure;

FIG. 55 is a perspective view of the jaw, in accordance with an aspect of the present disclosure;

FIG. 56 is a perspective view of the completed direct repair, in accordance with an aspect of the present disclosure;

FIG. 57 is a side view of the completed direct repair, in accordance with an aspect of the present disclosure;

FIG. 58 is a sectional view of the completed direct repair, in accordance with an aspect of the present disclosure;

FIG. 59 is a perspective view of a stepped diameter conduit, in accordance with an aspect of the present disclosure;

FIG. 60 is a side view of a stepped diameter conduit, in accordance with an aspect of the present disclosure;

FIG. 61 is a sectional view of a stepped diameter conduit, in accordance with an aspect of the present disclosure;

FIG. 62 is a top view of carrier for rolled ends conduit, in accordance with an aspect of the present disclosure;

FIG. 63 is a side view of carrier for rolled ends conduit, in accordance with an aspect of the present disclosure;

FIG. 64 is a sectional view of carrier for rolled ends conduit, in accordance with an aspect of the present disclosure;

FIG. 65 is a sectional view of carrier for rolled ends conduit, in accordance with an aspect of the present disclosure;

FIG. 66 is a perspective view of a short gap repair, in accordance with an aspect of the present disclosure;

FIG. 67 is a side view of a short gap repair, in accordance with an aspect of the present disclosure;

FIG. 68 is a sectional view of a short gap repair, in accordance with an aspect of the present disclosure;

FIG. 69 is a perspective view of a scar trimmer pull cut blade, in accordance with an aspect of the present disclosure;

FIG. 70 is a perspective view of a lever carrier housing with a separator, in accordance with an aspect of the present disclosure;

FIG. 71 is a side view of a lever carrier with a separator, in accordance with an aspect of the present disclosure;

FIG. 72 is an enlarged view of a lever carrier with a separator, in accordance with an aspect of the present disclosure;

FIG. 73 is a perspective view of a completed long defect repair, in accordance with an aspect of the present disclosure;

FIG. 74 is a side view of a completed long defect repair, in accordance with an aspect of the present disclosure;

FIG. 75 is a sectional view of a completed long defect repair, in accordance with an aspect of the present disclosure;

FIG. 76 is a side view of a carrier support stand, in accordance with an aspect of the present disclosure;

FIG. 77 is a perspective view of a carrier support stand, in accordance with an aspect of the present disclosure;

FIG. 78 is a side view of a spiral conduit, in accordance with an aspect of the present disclosure;

FIG. 79 is a perspective view of a spiral conduit, in accordance with an aspect of the present disclosure;

FIG. 80 is a perspective view of a wrapped conduit with end tabs, in accordance with an aspect of the present disclosure;

FIG. 81 is a side view of a lever activated carrier, in accordance with an aspect of the present disclosure;

FIG. 82 is a perspective view of a lever activated carrier, in accordance with an aspect of the present disclosure;

FIG. 83 is an exploded, perspective view of a lever activated carrier, in accordance with an aspect of the present disclosure;

FIG. 84 is a top view of a lever activated carrier, in the release position, in accordance with an aspect of the present disclosure;

FIG. 85 is a sectional view of a lever activated carrier, in the release position, in accordance with an aspect of the present disclosure;

FIG. 86 is a side view of the conduit for a lever activated carrier, in accordance with an aspect of the present disclosure;

FIG. 87 is an end view of the conduit for a lever activated carrier, in accordance with an aspect of the present disclosure;

FIG. 88 is a perspective view of the conduit for a lever activated carrier, in accordance with an aspect of the present disclosure;

FIG. 89 is a side view of a simple conduit holder, in accordance with an aspect of the present disclosure;

FIG. 90 is a side view of a simple conduit holder and conduit, in accordance with an aspect of the present disclosure;

FIG. 91 is a sectional view along line 91-91 of FIG. 90, of a simple conduit holder and conduit, in accordance with an aspect of the present disclosure;

FIG. 92 is an enlarged view of a simple conduit holder and conduit, in accordance with an aspect of the present disclosure;

FIG. 93 is a perspective view of a multiple port conduit, in accordance with an aspect of the present disclosure;

FIG. 94 is a perspective view of a conduit with linked tabs, in accordance with an aspect of the present disclosure;

FIG. 95 is a perspective view of a conduit with twin vacuum ports, in accordance with an aspect of the present disclosure;

FIG. 96 is a perspective view of a conduit with a slit, in accordance with an aspect of the present disclosure;

FIG. 97 is a perspective view of a conduit with the dovetail tabs in the open position, in accordance with an aspect of the present disclosure;

FIG. 98 is a perspective view of a conduit with the dovetail tabs in the closed position, in accordance with an aspect of the present disclosure;

FIG. 99 is an end view of a conduit with the dovetail tabs in the open position, in accordance with an aspect of the present disclosure;

FIG. 100 is an end view of a conduit with the stepped tabs in the open position, in accordance with an aspect of the present disclosure;

FIG. 101A is a flow diagram of a method of performing a nerve repair procedure, in accordance with aspects of the present disclosure; and

FIG. 101B is a continuation of the flow diagram of the method of FIG. 101A, in accordance with aspects of the present disclosure.

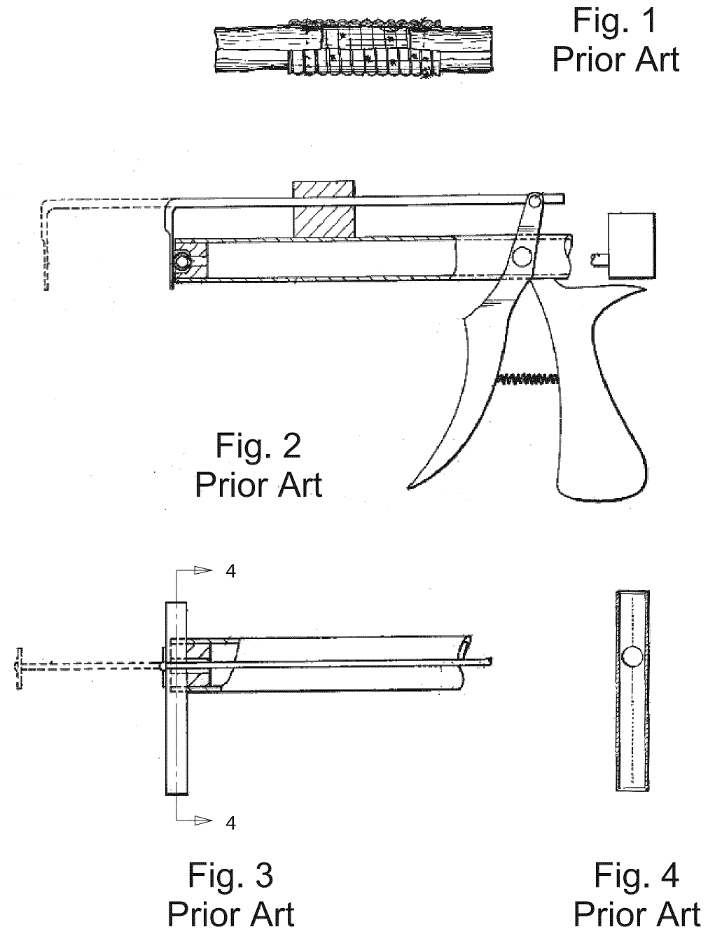

### DETAILED DESCRIPTION OF THE INVENTION

Certain examples will now be described to provide an overall understanding of the principles of the structure, function, manufacture, and use of the methods, systems, and devices disclosed herein. One or more examples are illustrated in the accompanying drawings. Those skilled in the art will understand that the methods, systems, and devices specifically described herein and illustrated in the accompanying drawings are non-limiting examples and that the scope of the present disclosure is defined solely by the claims. The features illustrated or described in connection with one example may be combined with the features of other examples. Such modifications and variations are intended to be included within the scope of the present disclosure.

The terms “significantly”, “substantially”, “approximately”, “about”, “relatively,” or other such similar terms that may be used throughout this disclosure, including the claims, are used to describe and account for small fluctuations, such as due to variations in processing from a reference or parameter. Such small fluctuations include a zero fluctuation from the reference or parameter as well. For example, they can refer to less than or equal to ±10%, such as less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

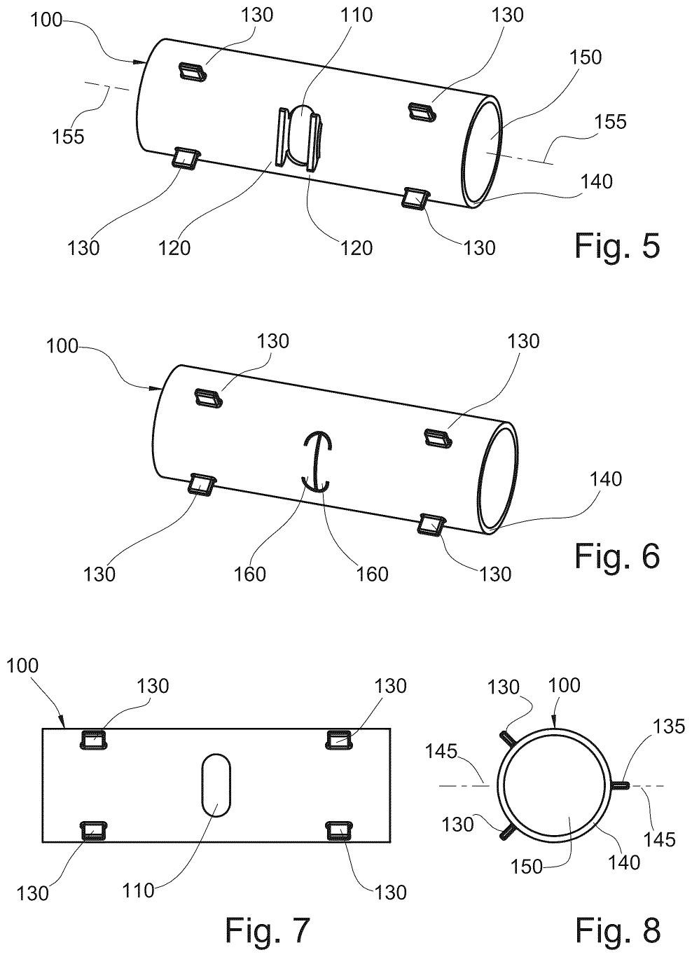

FIG. 5 is a perspective view of the conduit with fixed tabs and vacuum port flaps open. The conduit 100, is a generally cylindrical hollow tube, 140. It may be constructed from silicone, polymer, resorbable fiber, either solid or trabecular. The fabrication method can be molded. The conduit has an opening in the side, 110, a vacuum port, that is located approximately in the center of the length of the tube. The material that is adjacent to the port can be folded back to make a vacuum flap or flaps, which functions to keep growing axons from exiting the tube after implantation. There are a series of retaining tabs, 130, near the ends of the tube to maintain the tube's shape during vacuum coaptation. These are essential when the tube is made from thin, flexible material. The tab's width is from 0.2 to 3 millimeters, the tab's length parallel to the centerline is from 1 to 20 millimeters, and the tab height can vary from 0.5 to 5 millimeters. The preferred number of tabs adjacent to each end is three, but can vary from two to six.

The flaps adjacent to the vacuum port will be held open by the solid separator that is shown in later FIGS. The shape of the opening 150, is approximately circular, matching the sectional shape of a nerve stump. The conduit centerline 155 is the geometric axis of the conduit wall 140.

FIG. 6 shows the molded conduit, the same one in FIG. 5, however the vacuum port flaps are in the closed position, 160.

FIG. 7 shows the conduit 100 in the side view. Another embodiment of the vacuum port region is shown, one without flaps. The vacuum port 110 is between 1 and 5 millimeters tall and between 0.1 and 4 millimeters wide. The wall of the conduit will contain most of the advancing axons, and a relatively small opening such as the vacuum port 110, will not lose much of the benefit of the conduit.

FIG. 8 is an end view of the conduit, 100. The three retaining tabs 130 are approximately evenly spaced. The number of tabs can be between 2 and 6. The position of the three tabs maintains the shape of the nerve loading opening 150 during both when vacuum is applied to the instrument, and when it is not. The clamping plane 145 goes through the retaining tab centered 135 and the conduit centerline 155.

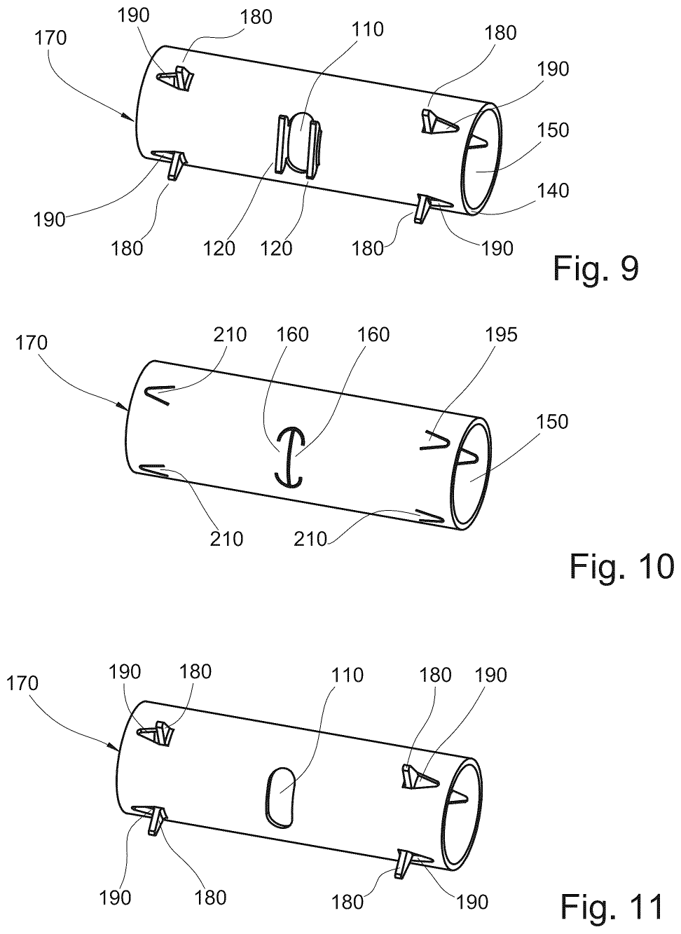

FIG. 9 is a perspective view of the conduit with folded tabs, 170, and vacuum port flaps open. The conduit 100, is a generally cylindrical hollow tube, 140. It may be constructed from silicone, polymer, resorbable fiber, either solid or trabecular. The fabrication method can be molded, electrodeposited, laser or die cut. The conduit has an opening in the side, 110, a vacuum port, that is located about in the center of the length of the conduit. The material that is adjacent to the port can be folded back to make a vacuum flap, which functions to keep growing axons from exiting the conduit after implantation. There are a series of protrusions, folded tabs, 180, near the ends of the conduit to maintain the conduit's shape during vacuum coaptation, the tabs are folded from the conduit wall leaving an opening 190. These openings are away from the axonal regrowth cavity. Their relatively small size does not impede the instrument's vacuum coaptation of the nerve stump. Folded tabs 180 are preferred when the conduit is made from hard to injection mold material. The shape of the opening 150, is approximately circular, matching the sectional shape of a nerve stump. The folded tabs width is from 0.2 to 3 millimeters, the length parallel to the centerline is from 1 to 20 millimeters. The preferred number of tabs is three, but can vary from two to six.

FIG. 10 is a perspective view of the conduit, 170, with the radial support tab, 195, in the relaxed or closed position. The vacuum flaps 160 are also in the closed position.

FIG. 11 is a perspective view of the conduit, 170, with the folded tabs 180 extended and the simple open vacuum port, 110.

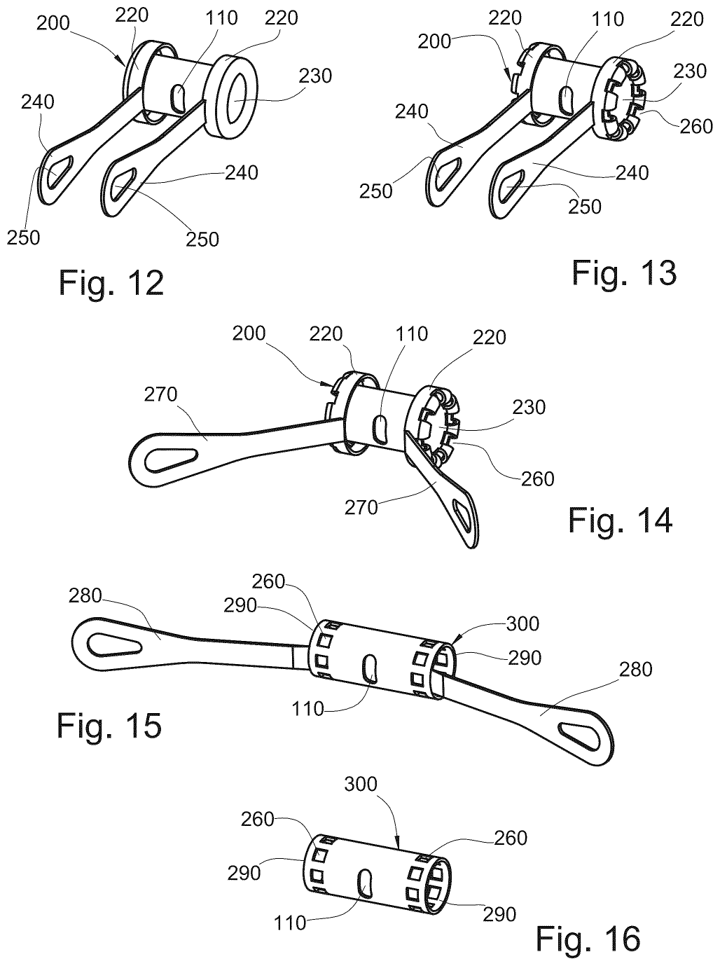

FIG. 12 is a perspective view of the conduit with rolled back ends 200 with the rolled back ends 220 providing radial support, and having a nerve entry region 230 that maintains its shape. The conduit with rolled ends, 200, has a vacuum port, 110. It has two unrolling tabs 240, to unroll the rolled back ends 220. The unrolling tabs 240, have openings 250, to maintain position during storage and instrument use. There is an unrolling tab 240, for each end of the conduit.

FIG. 13 is a perspective view of a conduit with rolled back ends 220. The wall of the conduit has openings 260 near each end. The openings 260 are spaced radially, typically between 4 and 12 places. The opening 260 allow the size and elasticity of the conduit to be tuned for ease of unrolling and contact force with nerve. Unrolling tabs 240 are used to unroll the rolled ends 220, and have retaining holes 250.

FIG. 14 is a perspective view of the conduit with rolled back ends 200, with the unrolling tabs 270 partially deployed.

FIG. 15 is a perspective view of the conduit with rolled back ends 200, with the unrolling tabs 280 fully deployed, and the unrolled conduits ends, 290, in their final position.

FIG. 16 is a perspective view of the rolled end conduit 300, with both of the unrolling tabs 280 trimmed off.

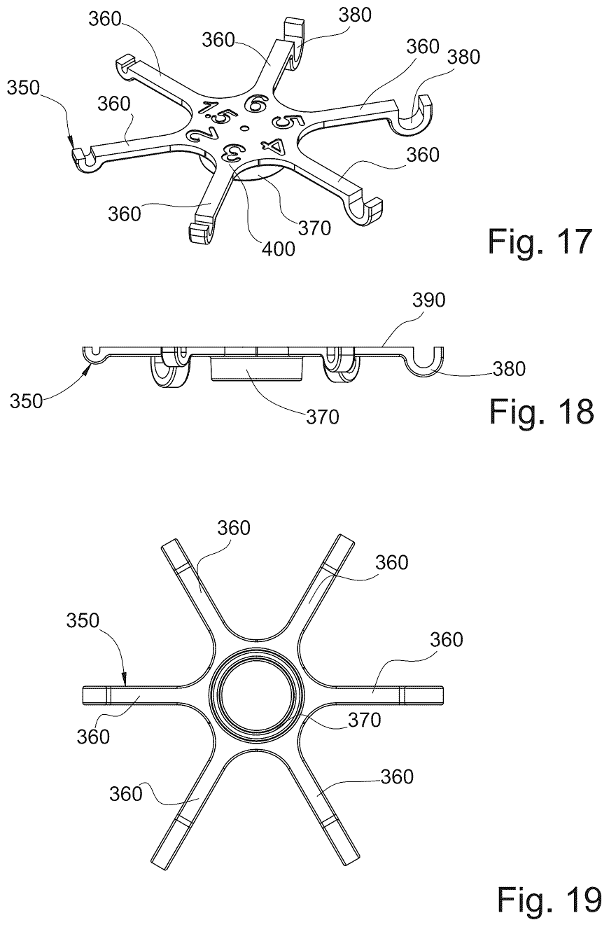

FIG. 17 is a perspective view of the minimally invasive nerve sizing instrument, 350. The minimally invasive nerve sizing instrument 350, has numerous arms 360, emanating from a hub 370. The arms 360 support a narrow U-shaped recess, 380. Each arm has remote sizing information 400 located on the central region.

FIG. 18 is a side view of the minimally invasive nerve sizing instrument, 350. The U-shaped recess, 380 and adjacent reference face 390 are visible. Each arm has a different dimensioned U-shaped recess, 380 with the increments being preferably 1 millimeters.

FIG. 19 is a bottom view of the minimally invasive nerve sizing instrument, 350. The hub 370 allows rotation between fingertips to align the proper arm 360 into place. The number of arms varies from 3 to 8.

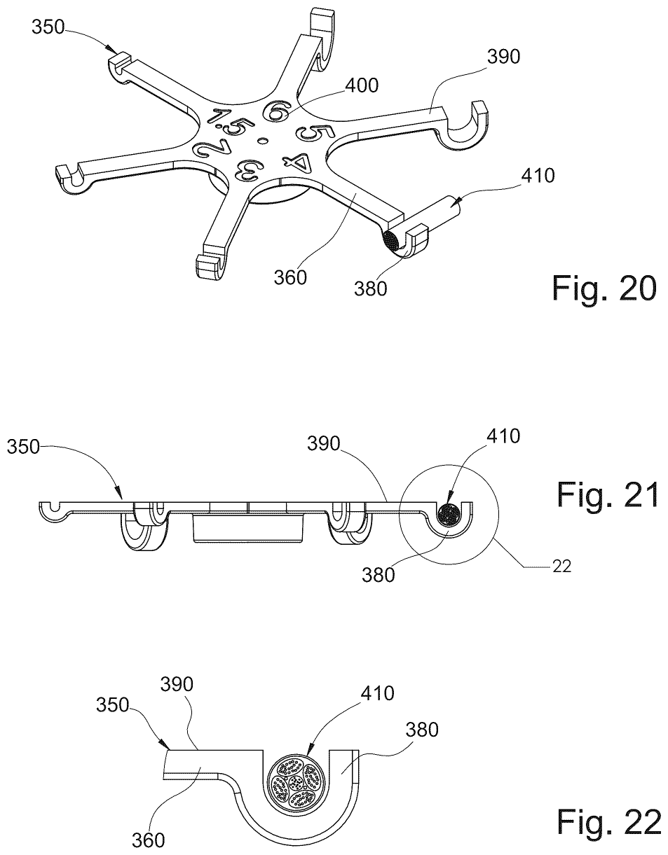

FIG. 20 is a perspective view of the minimally invasive nerve sizing instrument, 350, with a nerve stump 410 engaged in a U-shaped recess, 380.

FIG. 21 is a side view of the minimally invasive nerve sizing instrument, 350, with a nerve stump 410 engaged in the U-shaped recess, 380.

FIG. 22 is a detail side view of the minimally invasive nerve sizing instrument, 350, with a nerve stump 410 engaged in the U-shaped recess, 380. The tangency between the reference face 390 and the surface of the nerve stump 410 is the indication of proper recess size selection.

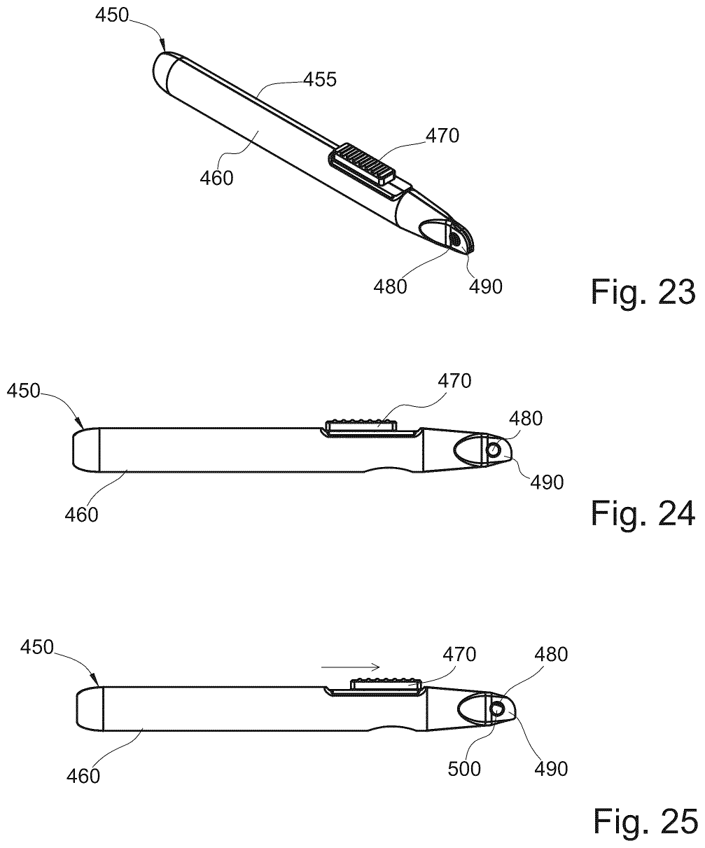

FIG. 23 is a perspective view of the scar trimmer, 450. The trimmer has a trimmer housing, 455 and a trimmer housing notched, 460, and activation button, 470, a forward narrow boss, 490, with a nerve stump cutting cavity, 480.

FIG. 24 is a side view of the scar trimmer 450 with the activation button 470 in the resting state. The stump cutting cavity 480 is open and unobstructed.

FIG. 25 is a side view of the scar trimmer 450 with the activation button 470 in the forward position. The stump cutting cavity 480 is not open, but the blade 500, if visible.

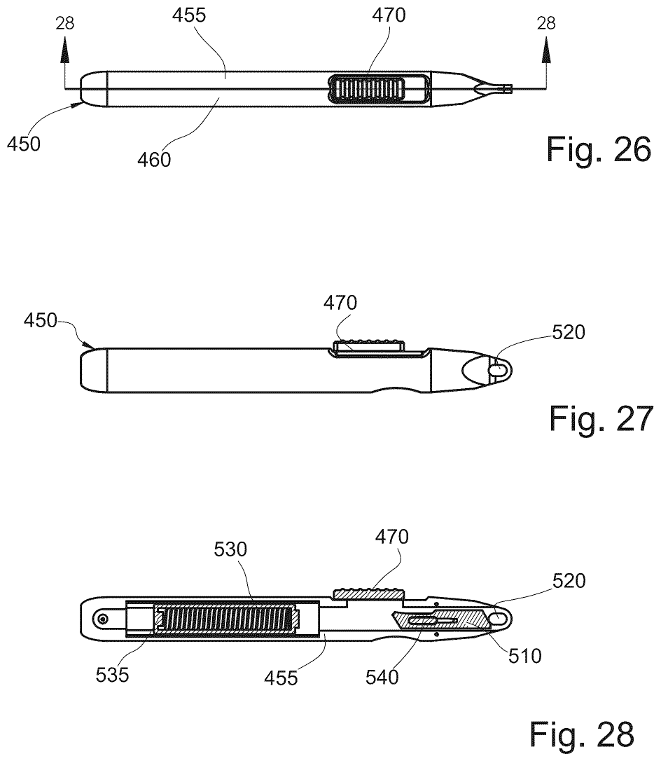

FIG. 26 is a top view of the scar trimmer 450, with a trimmer housing 455 and trimmer housing notched 460, and an activation button, 470.

FIG. 27 is a side view of the scar trimmer 450 with the activation button 470 in the resting position. The stump cutting cavity 520 is oval shaped.

FIG. 28 is a sectional view of the scar trimmer, 450. The activation button 470 has a spring capture loop 535, and a blade capture boss, 540, and a cutting blade with an oblique edge 510. The return spring, 530, is between the activation button spring loop 535 and a surface on the trimmer housing 455.

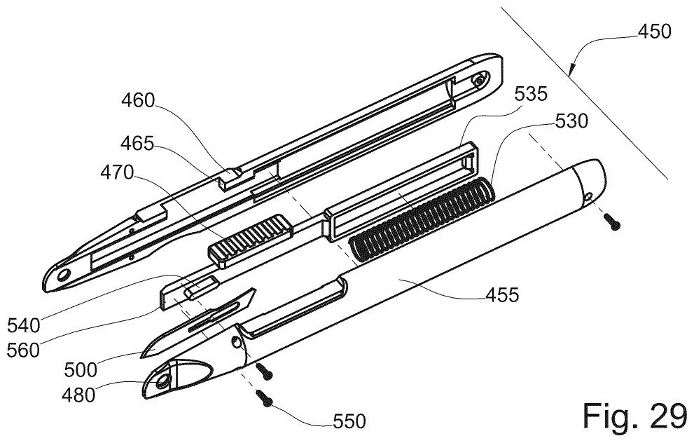

FIG. 29 is an exploded view of the scar trimmer, 450. The trimmer housing 455, and trimmer housing notched, 460, capture the internal slider 560 to which the activation button 470 is integral. The internal slide has a spring capture loop 535, to capture the return spring 530. The blade 500 fits over the blade capture boss 540. The activation button limit recess 465 limits travel of the activation button 470, internal slider 560, and blade 500. Fasteners 550 are used to hold the trimmer housing 455 and trimmer housing notched 460 and other components in place.

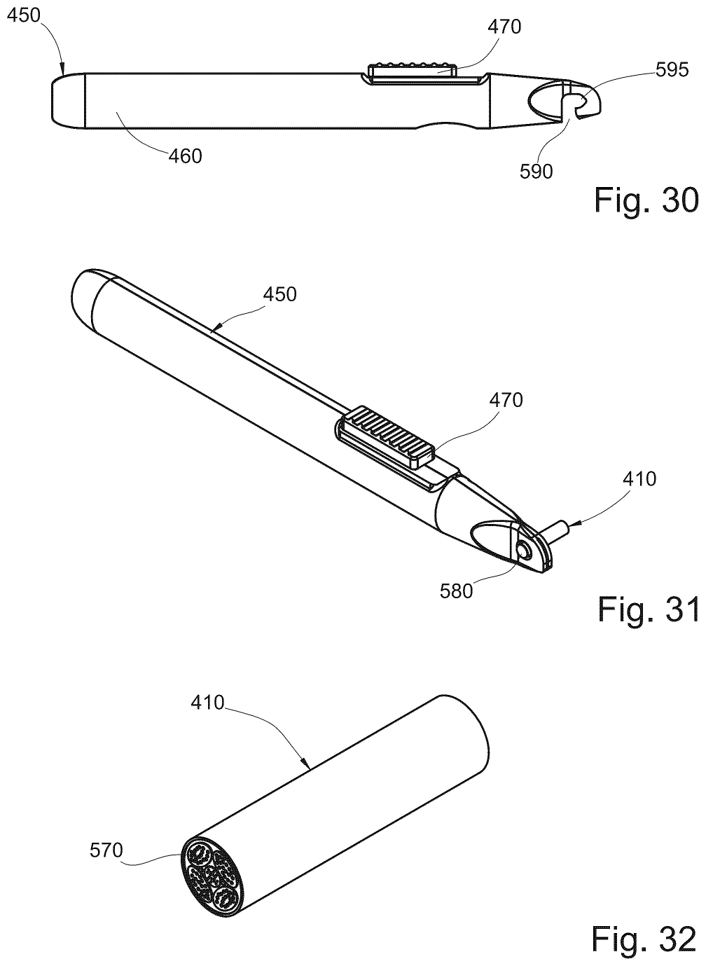

FIG. 30 is a side view of the scar trimmer with an open stump cutting recess 590. The vee shaped recess 595 of the stump cutting access slot 590 is used for smaller nerves.

FIG. 31 is a perspective view of a scar trimmer 450 with a nerve stump 410 in cutting position. The scar 580 on the nerve stump end is visible.

FIG. 32 is a perspective view of a nerve stump 410 that has had the scar trimmed off, leaving the viable fascicles 570 exposed.

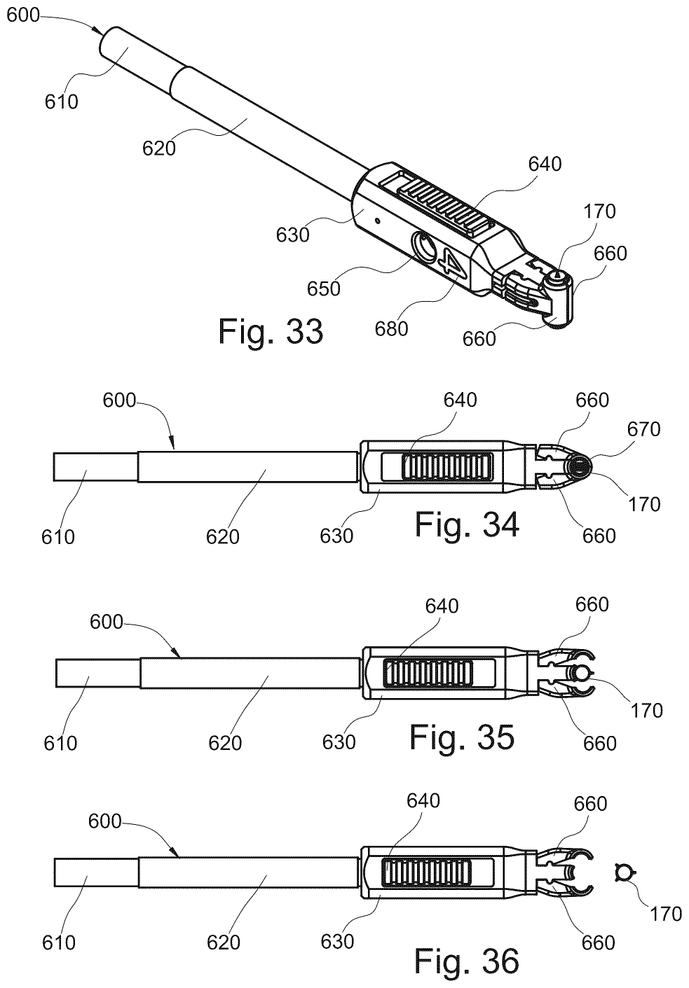

FIG. 33 is a perspective view of the conduit carrier, 600. The conduit carrier central housing 630 is the main housing. The vacuum connection cylinder 610 is fit into the conduit carrier central housing 630. There is a silicone sleeve 620 over the vacuum connection cylinder 610. The vacuum control port 650 is through one side of the conduit carrier central housing 630. There is conduit sizing text 680 on the instrument. The release button 640 is on another side. There are a pair of jaws 660 holding the conduit with folded tabs 170.

FIG. 34 is a side view of the conduit carrier 600 with the jaws 660 in the closed position. The release button 640 is closer to the jaws 660. The conduit with folded tabs 170 is fully surrounded by the conduit carrier central housing 630 and the jaws 660. The separator and jaw spring 670 is crossing the interior of the conduit with folded tabs 170.

FIG. 35 is a side view of the conduit carrier 600 with the jaws 660 open, ready to disengage the conduit with folded tabs 170. Both of the jaws 660 are rotated away from the conduit with folded tabs 170. The release button 640 has been moved away from the conduit with folded tabs 170.

FIG. 36 is a side view of the conduit carrier 600 with the jaws 660 open and the conduit carrier 600 withdrawn from the conduit with folded tabs 170.

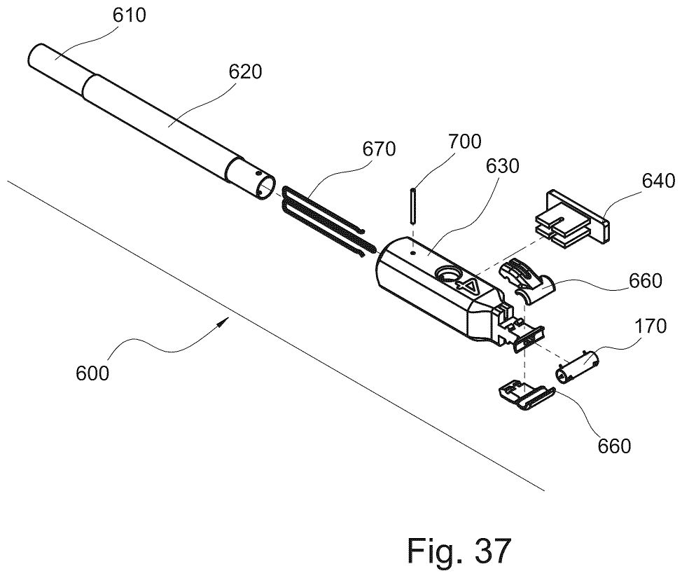

FIG. 37 is an exploded view of the conduit carrier 600. The separator and jaw spring 670 passes into the conduit carrier central housing 630, exiting into the jaws 660. The conduit with folded tabs 170 is between the jaws 660. The release button 640 is inserted into the conduit carrier central housing 630, and interlocks with the separator and jaw spring 670. The vacuum connection cylinder 610 and silicone sleeve 620 are loaded into the conduit carrier central housing 630 and held in place with the retention pin 700.

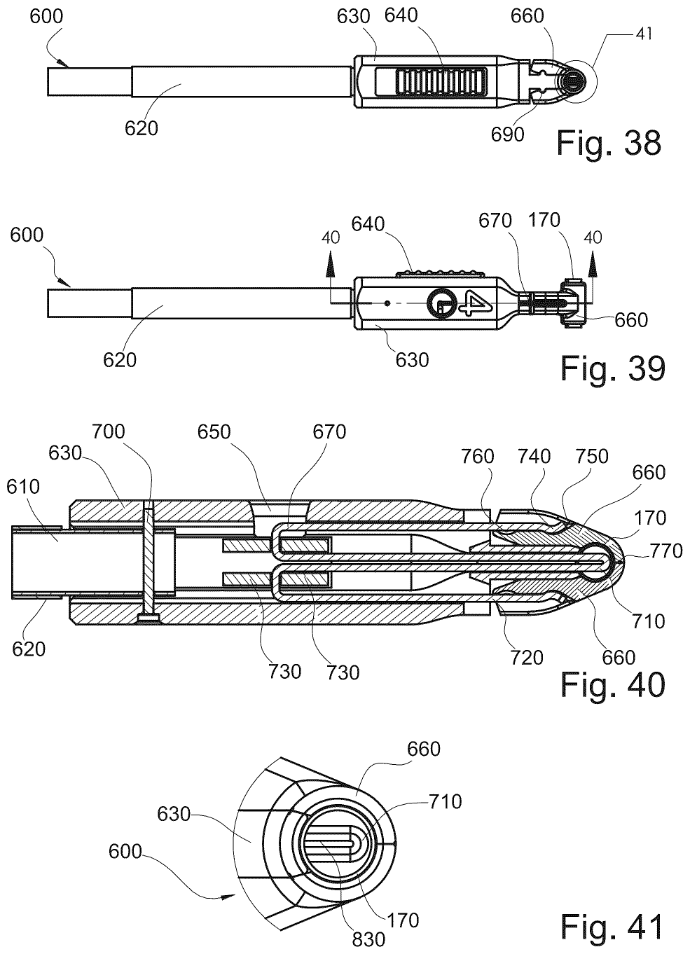

FIG. 38 is a side view of the conduit carrier 600 with the jaws 660 in the closed position. The jaw hinge bosses 690 fit into the jaws 660.

FIG. 39 is a top view of the conduit carrier 600 with the jaws 660 in the closed position. The separator and jaws spring 670 is visible in the center of the jaw 660. The conduit with folded tabs 170 extends past the jaw 660 on both sides.

FIG. 40 is a sectional view of the conduit carrier 600 with the jaws 660 in the closed position. The vacuum connection cylinder 610 is held into conduit carrier central housing 630 with retaining pin 700. The separator and jaw spring 670 has a spring wire bend 740 at each free end. In the closed position, the spring wire bend 740 rests in a detent for closed position 750 of each jaw 660. The separator and jaw spring 670 passes through the release button spring boss 730. The jaws 660 have interlocking features 770 at their mating surface. The conduit with folded tabs 170 is bounded by the conduit carrier central housing 630 and the jaws 660.

FIG. 41 is a close-up, side view of the conduit carrier 600 showing the separator loop 710 portion of the separator and jaw spring 670 is within the conduit with folded tabs 170. The intra separator space 830 is inside the separator loop 710.

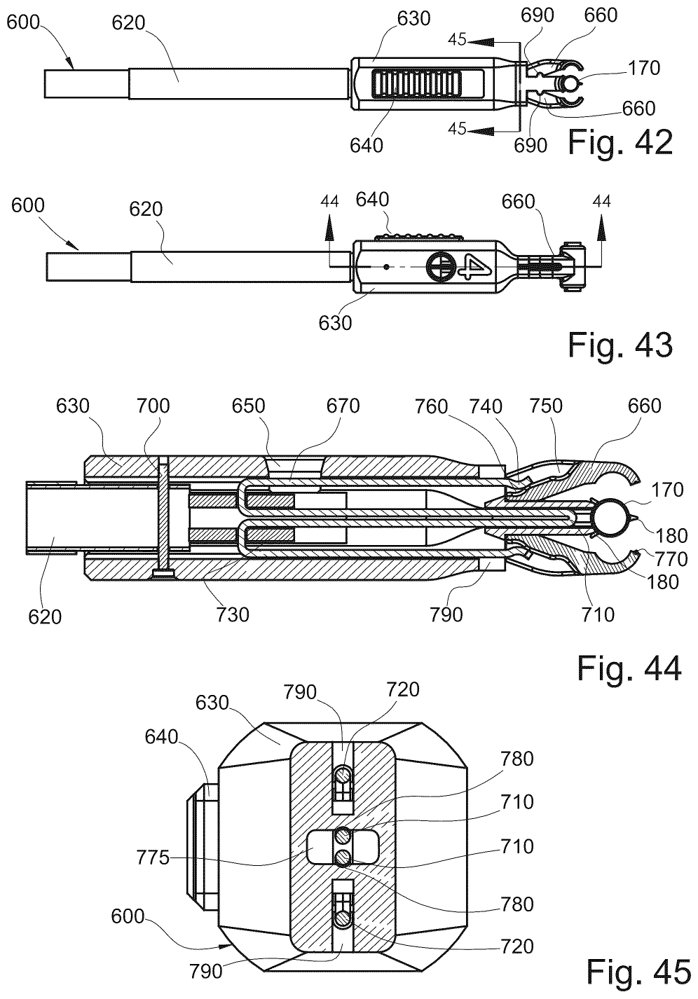

FIG. 42 is a side view of the conduit carrier 600 with the jaws 660 in the open position. The release button 640 is in a pulled back position. The conduit with folded tabs 170 is exposed. The jaw hinge boss 690 is visible on each jaw 660 about which rotation occurs.

FIG. 43 is a top view of the conduit carrier 600 with the jaws 660 in the open position.

FIG. 44 is a sectional view of the conduit carrier 600 with the jaws 660 in the open position. The release button spring boss 730 has been moved to the retracted position in contact with the vacuum connection cylinder 610. The separator and jaw spring 670 in contact with release button spring boss 730 has been moved to the retracted position. The spring wire bend 740 is in the detent for open position 760. The separator loop 710 is outside of the conduit with folded tabs 170. Conduit with folded tabs 170 and its folded tabs 180 are not retained by the jaws 660. The stabilizing interlocking boss 770 are not blocking the conduit with folded tabs 170 exit.

FIG. 45 is a sectional view of the conduit carrier 600. The conduit carrier central housing 630 has a spring clearance recess 790 with cantilever beam spring 720 passing through on both sides. The interior vacuum channel 775 is for vacuum through the body of conduit carrier central housing 630. The interior vacuum channel 775 has a pair of spring loop capture notches 780 for holding separator loop 710 with one degree of freedom.

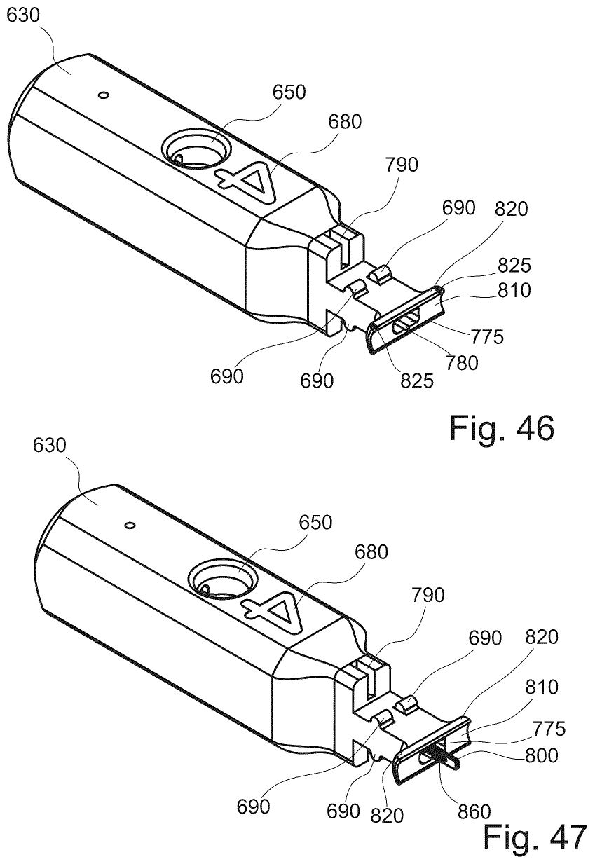

FIG. 46 is a perspective view of the conduit carrier central housing 630 for mobile separator. The vacuum control port 650 and conduit sizing text 680 are on one face of the conduit carrier central housing 630. There are two spring clearance recesses 790. There are four jaw hinge bosses 690 and it has a conduit contact face 810, with two adjacent tab pinch faces 820. The interior vacuum channel 775 has a pair of spring loop capture notches 780. There are two tab capture recesses 825 on each tab pinch face 820.

FIG. 47 is a perspective view of the conduit carrier central housing 630 with a fixed separator 800. The vacuum control port 650 and conduit sizing text 680 are on one face of the conduit carrier central housing 630. There are two spring clearance recesses 790. There are four jaw hinge bosses 690 and it has a conduit contact face 810, with two adjacent tab pinch faces 820. The interior vacuum channel 775 in in the middle of conduit carrier central housing 630. Adjacent to conduit contact face 810 is a fixed separator 800, with a separator vacuum channel 860.

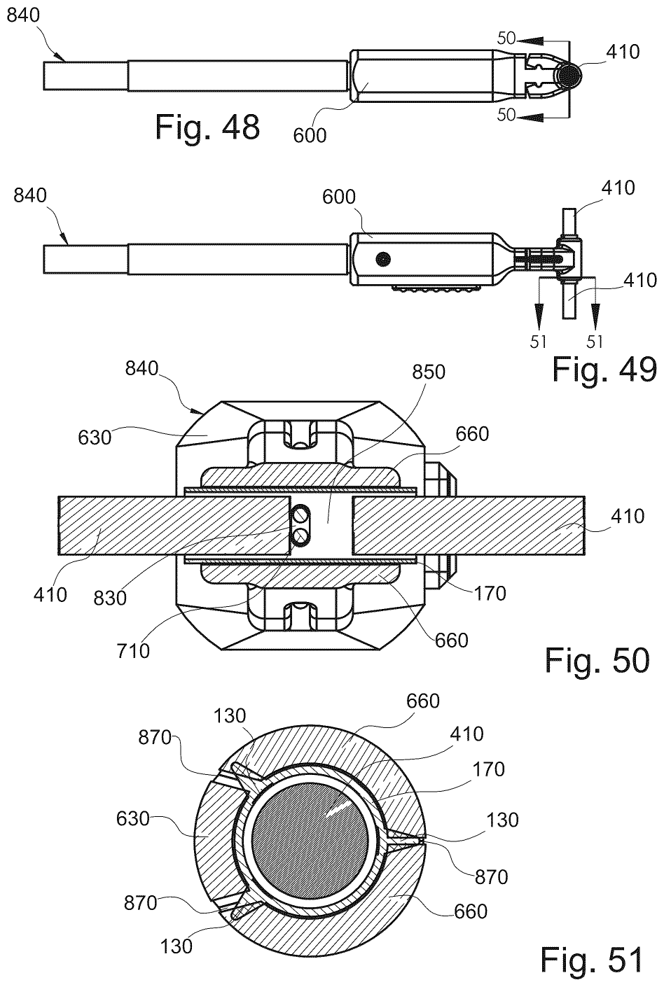

FIG. 48 is a side view of the conduit carrier engaged with two nerve stumps 840, the conduit carrier 600 with two nerve stumps 410.

FIG. 49 is a bottom view of the conduit carrier engaged with two nerve stumps 840, the conduit carrier 600 with two nerve stumps 410.

FIG. 50 is a sectional view of the conduit carrier with two nerves 840. The nerve stump 410 on the left is fully seated in the conduit with folded tabs 170, and is in contact with separator loop 710. The nerve stump 410 on the right is partially reduced, between the nerve 410 and the separator loop 710 is a low-pressure region 850. There is an intra separator space 830 in the center of the separator loop 710.

FIG. 51 is a sectional view of the jaws 660 holding the conduit with folded tabs 170. The retaining tabs 130 are pinched in three places, once between the jaws 660 and jaws 660, and in two places in between conduit carrier central housing 630 and jaws 660 in the tab capture recess 870. The nerve stump 410 is inside the conduit with folded tabs 170.

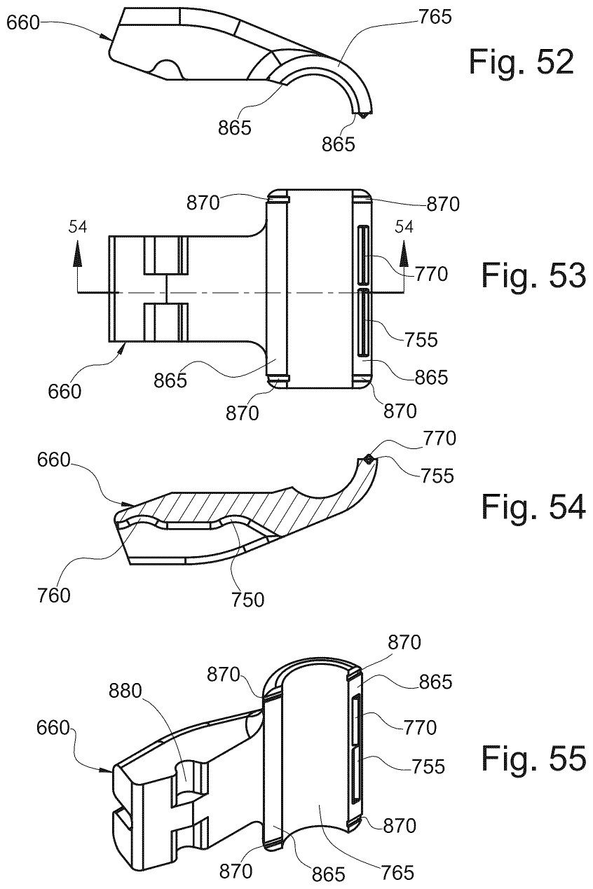

FIG. 52 is a side view of the jaw 660. Both of the jaw contact faces 865 are shown. The partial cylindrical boss 765 is seen in profile.

FIG. 53 is a bottom view of the jaw 660. There are several tab capture recesses 870 on each of the jaw contact faces 865. The jaw contact face 865 has a stabilizing interlocking boss 770 and stabilizing interlocking recess 755. The stabilizing interlocking recess 755 features are optional.

FIG. 54 is a section view of the jaw 660. There two recesses, a detent for closed position 750 and a detent for open position 760. The stabilizing interlocking boss 770 and stabilizing interlocking recess 755 are shown in profile.

FIG. 55 is a perspective view of the jaw 660. There is a recess on either side that is a hinge recess 880. One each jaw contact face 865 there are tab capture recesses 870 on either end of the partial cylindrical boss 765. The jaw contact face 865 has a stabilizing interlocking boss 770 and stabilizing interlocking recess 755.

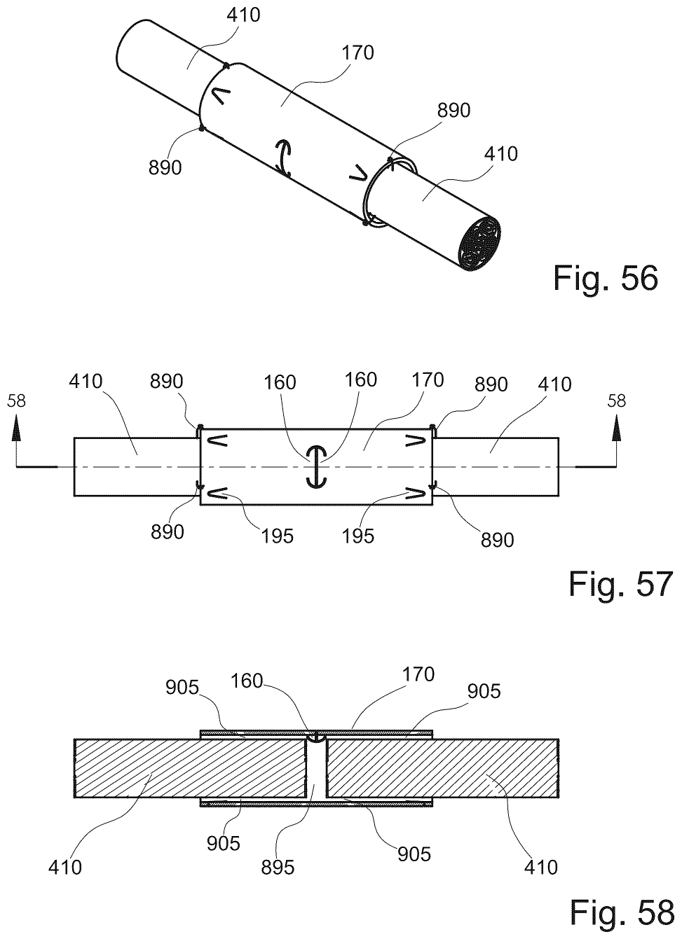

FIG. 56 is a perspective view of the conduit with folded tabs 170 with nerve stumps 410. There are sutures 890 to connect the conduit with folded tabs 170 to each of the nerve stumps 410.

FIG. 57 is a side view of the conduit with folded tabs 170 with two nerve stumps 410. The closed flaps 160 and retaining flaps neutral state 195 are folded flat into the cylindrical exterior surface of the conduit with folded tabs 170. The optional sutures 890 are shown spaced around the end of the conduit with folded tabs 170.

FIG. 58 is a sectional view of the conduit with folded tabs 170 with two nerve stumps 410. The space between the nerve stump 410 is the regeneration gap 895. The radial gap 905 is shown adjacent to each nerve stump 410.

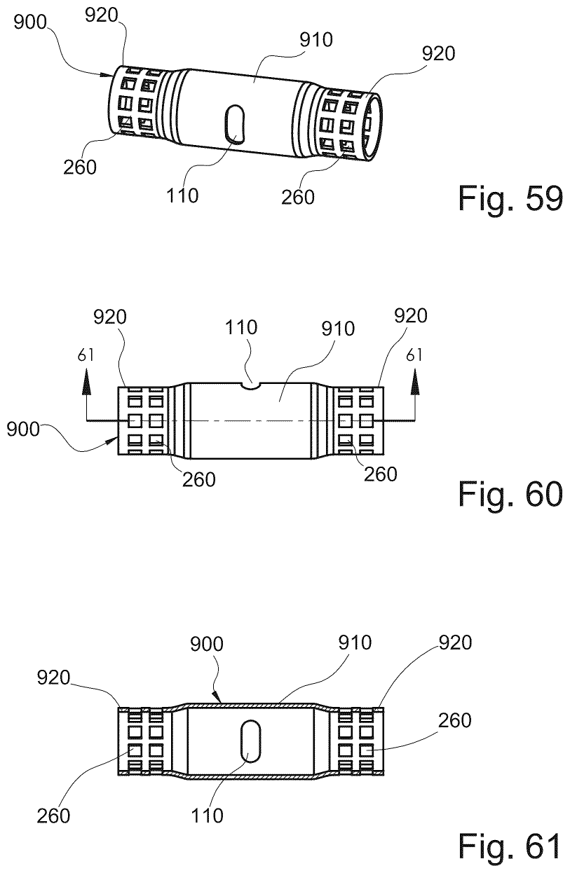

FIG. 59 is a perspective view of a stepped diameter conduit 900. The central segment of conduit 910 is flanked by the reduced diameter ends, 920. There is a vacuum port 110 is generally in the middle of the center section of conduit, 910. There may be a series of openings for elasticity tuning of conduit, 260, on the reduced diameter end, 920. The conduit stepped diameter 900 can be made without elasticity tuning of conduit 260.

FIG. 60 is a side view of a stepped diameter conduit 900. The center section of conduit 910 is flanked by the reduced diameter end 920. There is a vacuum port 110 is generally in the middle of the center section of conduit 910. There may be a series of openings for elasticity tuning of conduit 260 on the reduced diameter end 920.

FIG. 61 is a sectional view of a stepped diameter conduit 900. The center section of conduit 910 is flanked by the reduced diameter end 920. There is a vacuum port 110 is generally in the middle of the center section of conduit 910. There may be a series of openings for elasticity tuning of conduit 260 on the reduced diameter end 920.

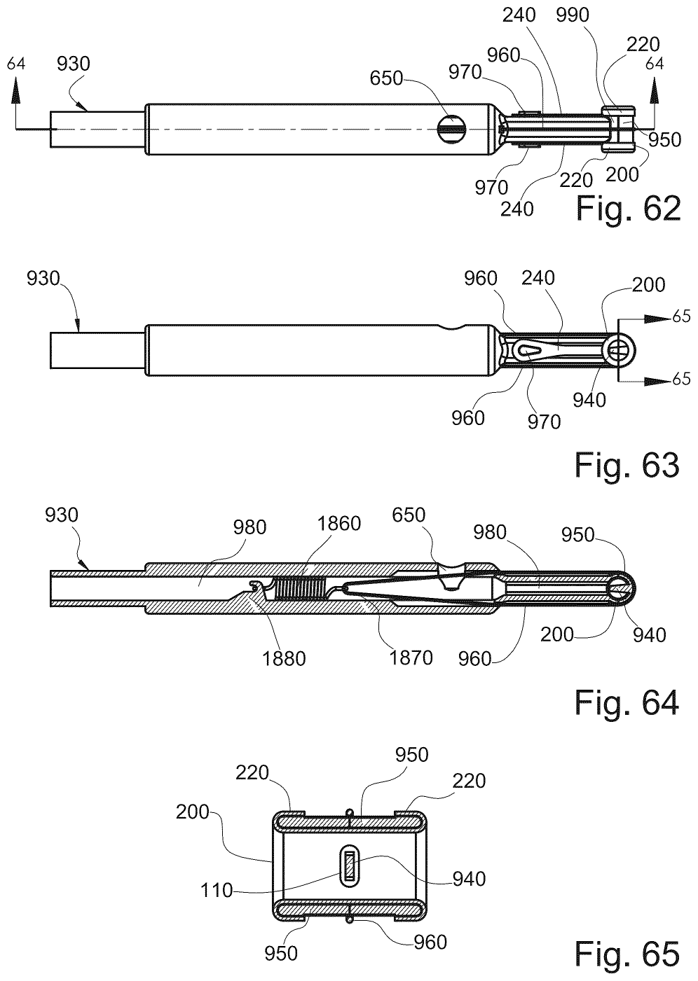

FIG. 62 is a top view of carrier for rolled end conduit 930. The carrier for rolled end conduit 930 has a partial cylindrical surface 990. The combination of partial cylindrical surface 990 and floating jaw 950 make a complete cylindrical boss about which the rolled back ends 220, of the conduit with rolled edges 200 is wrapped. The unrolling tabs 240 are held in place with the unrolling tab holding bosses 970. A flexible retainer 960 goes around the partial cylinder surface 990 and floating jaw 950. The vacuum control port 650 is on the top.

FIG. 63 is a side view of carrier for rolled ends conduit 930. The unrolling tabs 240 are held in place via unrolling tab holding boss 970. A flexible retainer 960 is used to hold partial cylindrical surface 990 to floating jaw 950 tightly, which holds conduit with rolled edges 200 open.

FIG. 64 is a sectional view of carrier for rolled ends conduit 930. Floating jaw 950 has a reversed separator 940 within the conduit with rolled edges 200. There is a vacuum channel 980 throughout the part. The flexible retainer 960 has a thread loop 1870, that is held in tension by retaining spring 1860, which is anchored by spring hook 1585.

FIG. 65 is a sectional view of carrier for rolled ends conduit 930. The rolled back ends 220 are wrapped back around the ends of the floating jaw 950. The reversed separator 940 passes through the vacuum port 110 of conduit with rolled edges 200. The flexible retainer 960 passes around the floating jaw 950.

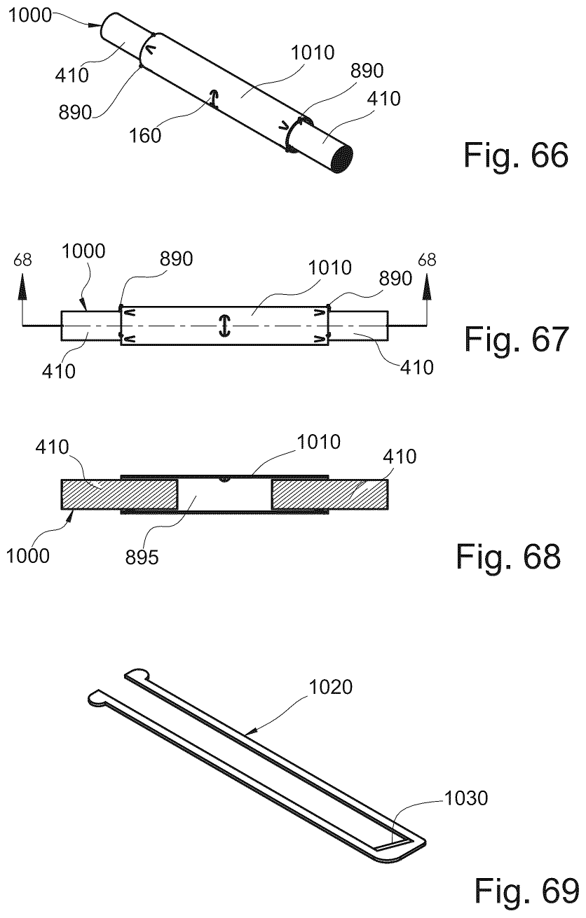

FIG. 66 is a perspective view of a large gap repair construct 1000. There are nerve stumps 410, that are partially inside a large gap conduit 1010. Anchoring sutures 890 are shown at the end of the large gap conduit 1010.

FIG. 67 is a side view of a large gap repair construct 1000. There are nerve stumps 410, that are partially inside a large gap conduit 1010. Anchoring sutures 890 are shown at the end of the large gap conduit 1010.

FIG. 68 is a sectional view of a large gap repair construct 1000. Between the nerve stump 410 nerves and nerve stump 410 nerves is the regeneration gap 895, in the large gap conduit 1010.

FIG. 69 is a perspective view of a pull cut blade 1020. The reverse cutting edge 1030 is shielded from the exterior of the pull cutting blade 1020.

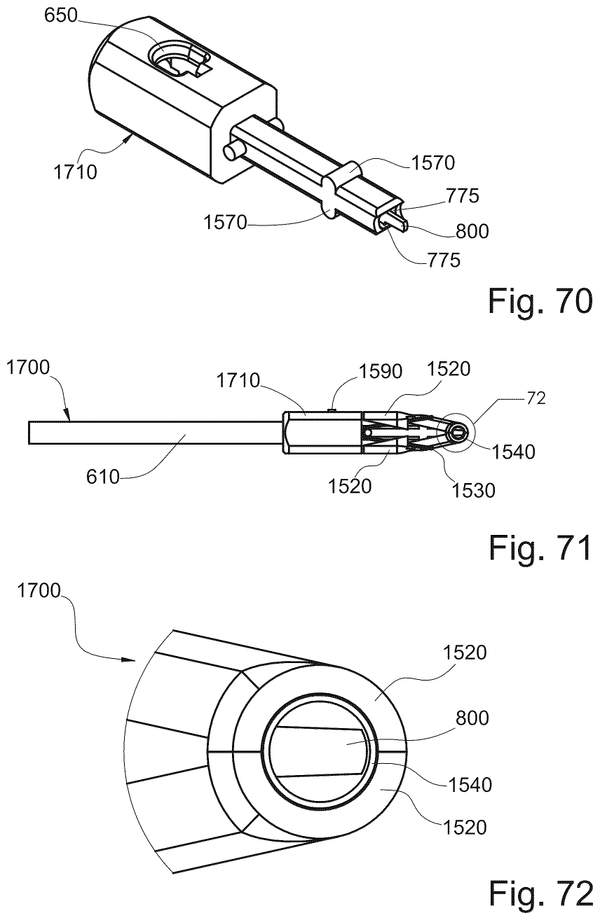

FIG. 70 is a perspective view of a lever carrier housing with a separator, 1710. It has an interior vacuum channel 775, a pair of jaw pivot bosses 1570, a vacuum control port 650, and the fixed separator 800.

FIG. 71 is a side view of a lever carrier with a separator, 1700. It has a lever activated carrier housing with a separator 1710, a pair of lever jaws 1520, a vacuum connection cylinder 610, a lever spring 1530, and a vacuum valve 1590, holding a conduit with two end tabs 1540.

FIG. 72 is a detail view of a lever carrier housing with a separator, 1700. It has a pair of lever jaws 1520, a conduit with two end tabs 1540, within which is the fixed separator 800.

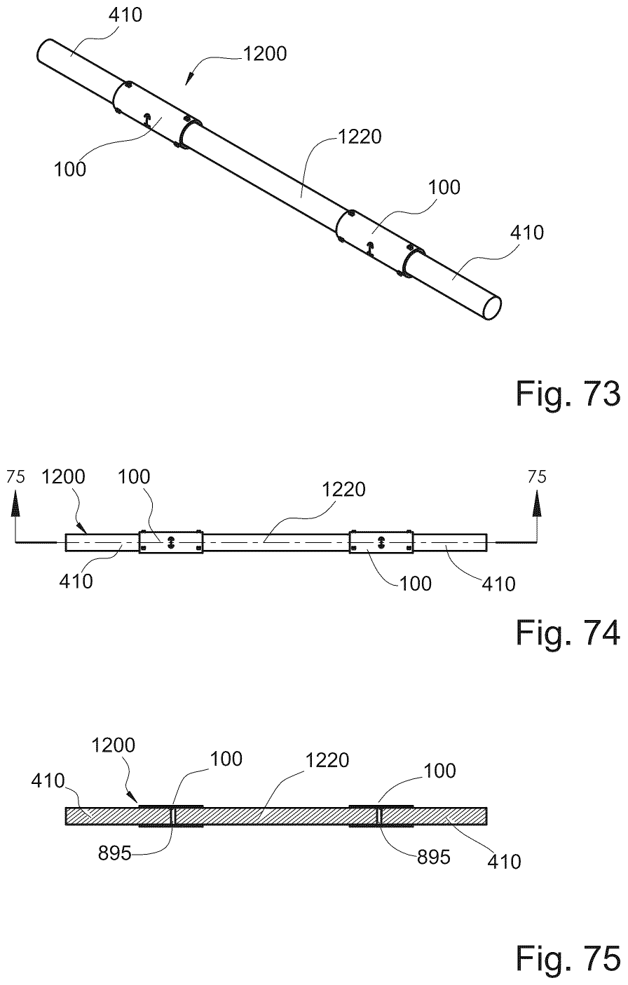

FIG. 73 is a perspective view of the completed long defect repair 1200. The nerve stumps 410 are partially inside each conduit 100. Each conduit 100 also envelops one end of the nerve allograft 1220.

FIG. 74 is a side view of the completed long defect repair 1200. The nerve stumps 410 are partially inside each conduit 100. Each conduit 100 also envelops one end of the nerve allograft 1220.

FIG. 75 is a sectional view of the completed long defect repair 1200. The nerve stumps 410 are partially inside each conduit 100. Each conduit 100 also envelops one end of the nerve allograft 1220. Within the conduit is the regeneration gap 895 on either side of the nerve allograft 1220.

FIG. 76 is a side view of a carrier stand 1300. There is a carrier tube recess 1310, that has a carrier tube feed channel, 1330. It has a carrier stand base 1320.

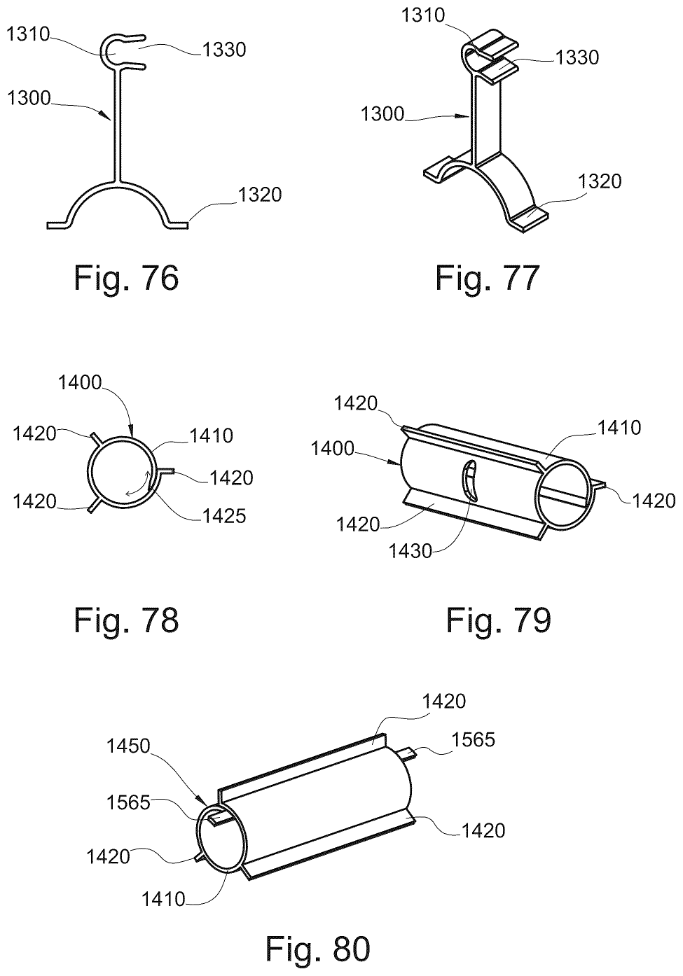

FIG. 77 is a perspective view of a carrier stand 1300. There is a carrier tube recess 1310, that has a carrier tube feed channel, 1330. It has a carrier stand base 1320.

FIG. 78 is an end view of a wrap conduit 1400. The wrap conduit 1400 has a spiral wall 1410, and at least one continuous support tab, 1420. The internal spiral end 1425 overlaps the spiral wall 1410.

FIG. 79 is a perspective view of a wrap conduit 1400. The wrap conduit 1400 has a spiral wall 1410, and at least one continuous support tab, 1420. There is a vacuum port 1430 through the spiral wall 1410.

FIG. 80 is a perspective view of a wrapped conduit 1400 with two spiral end tab 1565. It has a spiral wall 1410 and at least one continuous support tab 1420.

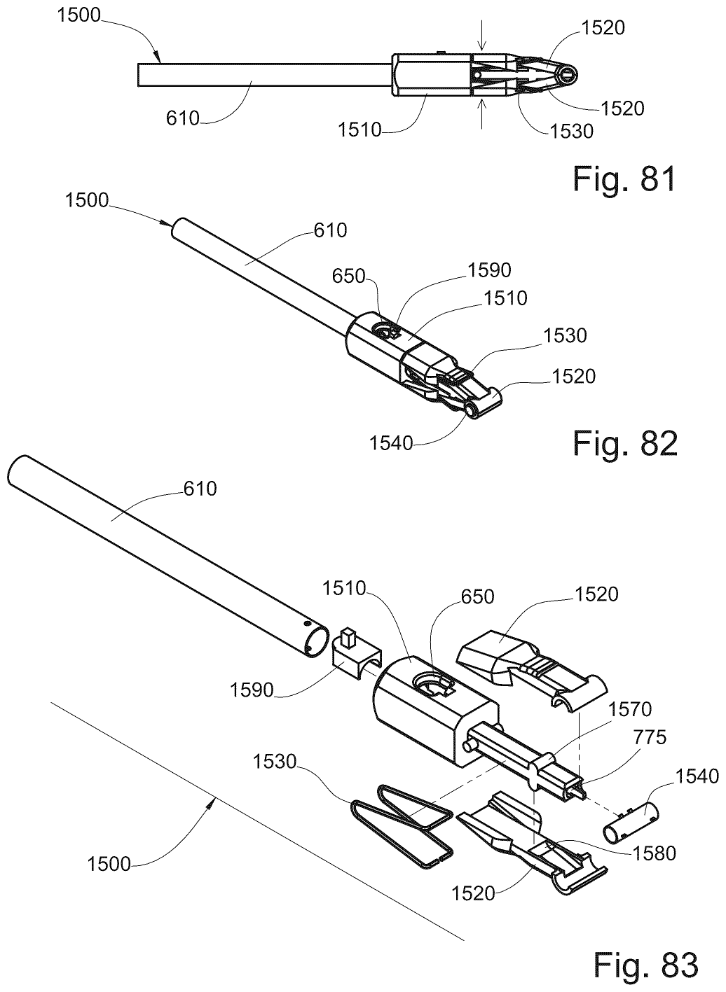

FIG. 81 is a side view of a lever activated carrier, 1500. It consists of a vacuum connection cylinder 610 and a lever activated carrier housing, 1510. It has a pair of lever jaws, 1520 and a lever spring, 1530.

FIG. 82 is a perspective view of a lever activated carrier, 1500. It consists of a vacuum connection cylinder 610 and a Lever activated carrier housing, 1510. It has a pair of lever jaws, 1520 and a lever spring, 1530 and a slidable vacuum valve 1590. The lever activated carrier 1500 holds a conduit with at least two end tabs 1540 or any of several other embodiments of carriers, e.g., conduits 100, 170, 1900.

FIG. 83 is an exploded view of a lever carrier 1500. It consists of a vacuum connection cylinder 610 and a lever activated carrier housing, 1510. It has a pair of lever jaws, 1520 and a lever spring, 1530. It has a vacuum control port 650, with a slidable vacuum valve 1590. It holds conduit with two end tabs, 1540. The interior vacuum channel 775 is visible inside lever activated carrier, 1510. Lever activated carrier housing, 1510 also has two jaw pivot bosses 1570. The lever jaws 1520 have jaw pivot hinge recess 1580. A mobile separator could be adapted to this carrier.

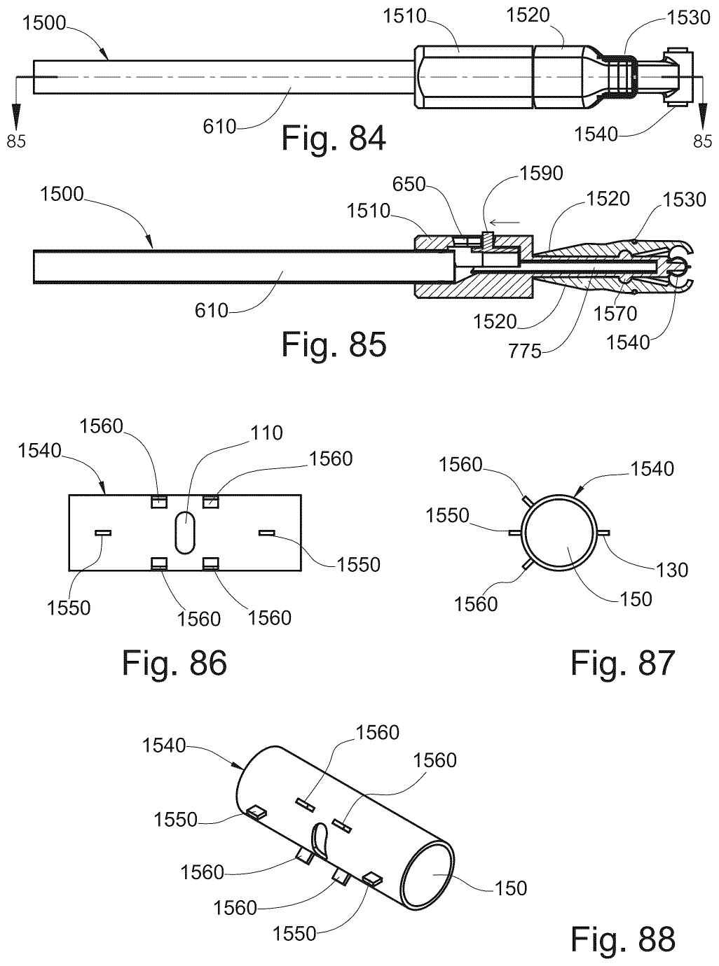

FIG. 84 is a top view of a lever activated carrier, 1500. It consists of a vacuum connection cylinder 610 and a lever activated carrier housing, 1510. It has a pair of lever jaws, 1520 and a lever spring, 1530. It holds conduit for conduit with two end tabs, 1540.

FIG. 85 is a sectional view of a lever carrier 1500. It consists of a vacuum connection cylinder 610 and a lever activated carrier housing, 1510. It has a pair of lever jaws, 1520 and a lever spring, 1530. It has a vacuum control port 650, with a slidable vacuum valve 1590. It holds conduit with two end tabs, 1540. The interior vacuum channel 775 is visible inside lever activated carrier housing 1510. Lever activated carrier housing 1510 also has jaw pivot boss 1570. lever jaws 1520 are in the release position.

FIG. 86 is a side view of the conduit for conduit 1540 with two end tabs 1550. Conduit 1540 has a vacuum port, 110. Near each open end 150, end tabs 1550 are spaced 180 degrees apart, i.e., positioned at 3 and 9 o'clock. Conduit 1540 also includes supporting tabs 1560 around the vacuum port 110. This is an alternate embodiment of tab arrangement and uses a jaw with multifaceted contact faces at 8 and 10 o'clock.

FIG. 87 is an end view of the conduit for conduit with two end tabs 1540. It has tab 180 degree, 1550, at the midline in two places at each end. It has tab port supporting (or supporting tabs) 1560 on one side and retaining tabs 130 on the other. The open end for nerve stump 150 is in its center.

FIG. 88 is a perspective view of the conduit for conduit with two end tabs 1540. It has a vacuum port 110. It has tab 180 degree 1550, at the midline. It has tabs port supporting 1560 around the vacuum port 110.

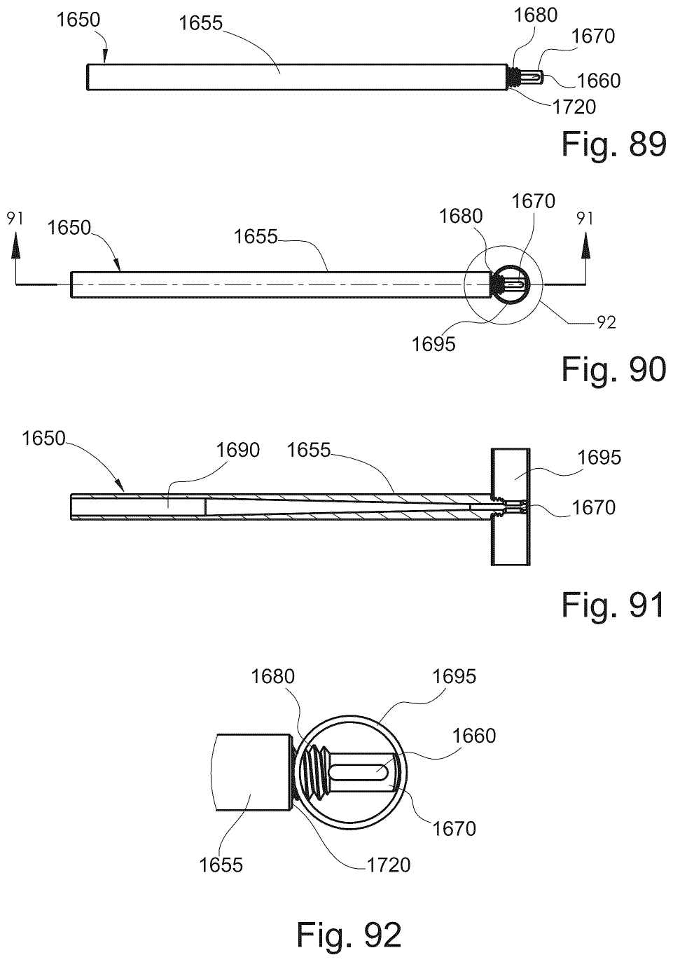

FIG. 89 is a side view of a simple conduit holder 1650. It has a shaft handle 1655, a simple separator 1670, a vacuum opening 1660, and an expanded separator region 1680. The contact face 1720 is adjacent to the expanded separator region 1680.

FIG. 90 is a side view of a simple conduit holder 1650 and conduit with round vacuum port 1695, a simple separator 1670, and an expanded separator region 1680.

FIG. 91 is a sectional view of a simple conduit holder 1650 and conduit with round vacuum port, 1695. The internal vacuum channel 1690 runs through the simple conduit holder handle 1655. The simple separator 1670 is within the conduit with round vacuum port 1695.

FIG. 92 is a detailed view of a simple conduit holder 1650 and conduit with round vacuum port 1695, a simple separator 1670, and an expanded separator region 1680. The contact face 1720 is adjacent to the expanded separator region 1680. The vacuum opening 1660 in the simple separator 1671 shown.

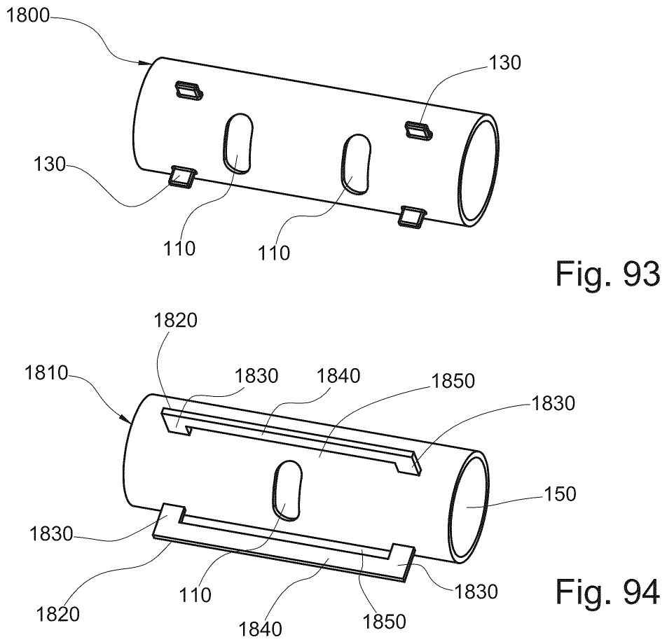

FIG. 93 is a perspective view of a multiple port conduit, 1800. It has more than one vacuum port, 110. It has numerous retaining tabs 130.

FIG. 94 is a conduit with linked tabs 1810. It has a vacuum port 110. Each linked tab 1820 has a radial tab segment 1830 on each end, and a linking segment 1840 connecting them, creating an open capture recess 1850.

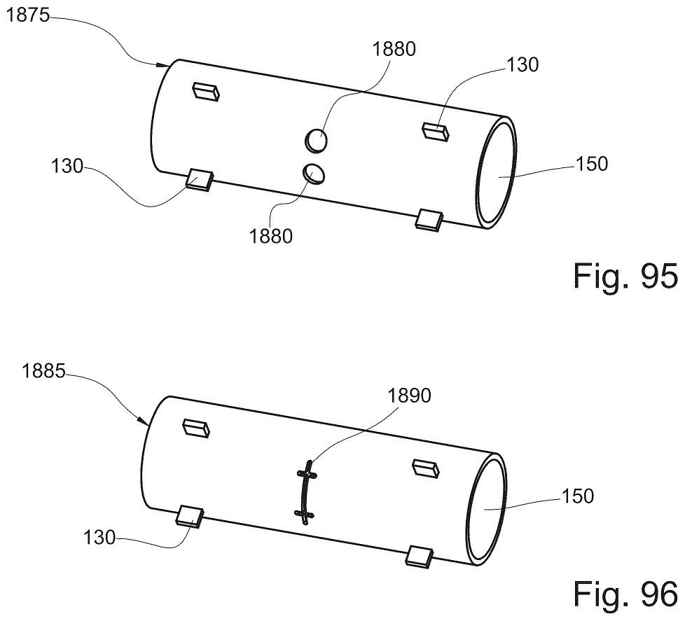

FIG. 95 is a perspective view of a conduit with twin vacuum ports. Conduit with small ports 1875 is a cylindrical conduit. There are two or more vacuum port small, 1880, that are sized for a twin post separator.

FIG. 96 is a perspective view of a conduit with a slit 1885. It is cylindrical and has a slit for a separator 1890. The slit will flex out of the way of separator.

FIG. 97 is a perspective view of a conduit with dovetail tabs in the open position. The conduit with dovetail tabs 1900, is cylindrical shaped implant. It has a series of dovetail tabs 1910, that are formed from a dovetail recess 1920, around the circumference. The conduit has an opening in the side, a vacuum port 110, that is located about in the center of the length of the conduit. Adjacent to this can be at least one vacuum port flap 120, to close it after implantation.

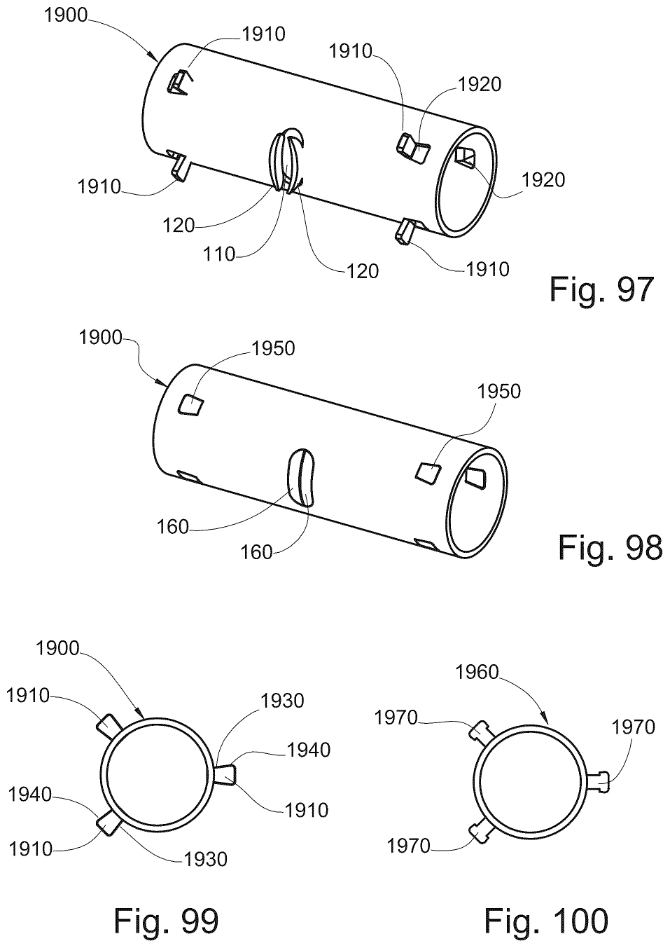

FIG. 98 is a perspective view of a conduit with dovetail tabs in the closed position. The conduit with dovetail tabs 1900, is in the as implanted condition, with the dovetail tabs folded in 1950. The vacuum port flaps 160 are shown.

FIG. 99 is an end view of a conduit with dovetail tabs in the open position 1900. It has a series of dovetail tabs 1910, and the transition from dovetail tab base 1930 to the larger dovetail tab end 1940, that allows the variable section tabs to interlock into the holding instrumentation.

FIG. 100 is an end view of a conduit with stepped tabs in the open position, 1960. It has a series of stepped tabs 1970, and allows the tabs to interlock into the holding instrumentation.

The conduits in FIGS. 5-11 are generally cylindrical in shape to capture the stumps of a severed nerve. The conduits, 100, 170, have vacuum ports 110 to create a low pressure region inside the conduit, drawing the nerve stumps 410 in without need for any other manipulative forces. The conduits have exterior bosses 130 that allows the support of the conduit so it does not collapse when the internal vacuum is applied. These tabs are spread out the exterior to provide balanced support. The tabs are pinched or interlocked into the carrier instrument 600, 50, to mechanically maintain the conduits cylindrical shape.

There is fixed tab 100 and folding tab 170 versions which can be made from various biocompatible materials. The material used for molding the conduit is silicone or another polymer. The cut tabs 180 are more adaptable to bioabsorbable fiber conduits, and can be die or laser cut. After cutting, they are folded outwards to provide contact with the instrument. The processing of bioabsorbable conduits often is only good at creating a uniform wall, and small features are not easily accommodated. The cutting is an economical and clean process. A fixture to push out the tabs could be employed during the loading operation of the instrument. The conduits are loaded into the instrument by the manufacturer and sterile packaged. The end user, the surgeon, will not need to perform this tedious loading operation.

The vacuum port 110 can have adjacent molded or cut out flaps 120. The instrument will hold these open during coaptation. They will naturally fall closed after instrument disengagement and can close off the port 160. The closed flaps will tend to keep advancing cells inside the conduit.

Conduits may have multiple vacuum ports 1800 to coapt the nerves with and without a separator. The vacuum port can be a matrix of small holes within the bounds of the vacuum port 110.

The conduits 100, 170 range in diameter from 1 to 10 mm, and in length from 5 to 100 mm. Longer length conduits will have a series of exterior support tabs 130 more than shown in the FIGS. 5-8 to maintain the cylindrical cross section.

Conduits shown in FIGS. 12-16 maintain the cylindrical shape by rolling the ends back 220 over a cylindrical holding instrument 930. They have vacuum ports 100, and because of the 360 degree support can be used with very flexible conduits, like those made from silicone 200. The rolled ends provide a vacuum-tight seal, The radius of the bend provides for a very easy loading as they act as centering ramps for the nerve stumps. To remove the conduit with rolled edges 200, integral unrolling tabs 240 are used. They rest against the instrument during implantation, then are pulled apart, pulling the edge out to its extended position. The natural position of the conduit is unrolled, and that is how they are molded, so it wants to return to this shape, in the same way a simple piece of hose would act. The tabs are trimmed off after the conduit is fully extended 300.

The rolled end conduit 300 can have openings 260 near the ends. These windows 260 make that portion of the conduit easier to roll back. They serve a secondary purpose in that they alter the stiffness of the conduit making it less likely the conduit will pinch the nerve if it is at a place where the nerve moves during skeletal ambulation, as nerves must flex with the body. These windows can be used with the non-rolled back conduits too. The windows can go all through the conduit wall, or partially.

The conduit can have all or part of the vacuum tube molded into it, and be trimmed off after implantation.

The sizing instrument 350 is shown in FIGS. 17-22. The sizer is a device that measures the diameter of a nerve stump. Consists of a U-shaped recess 380 that the nerve root 410 is inserted into. If the nerve falls in too loosely, it is undersized and if the nerve does not fit in the size is too large. It allows non optical scale, ruler, measurement of the nerve stumps. The arms 360 are narrow to allow use in small patients and in small wounds, making the repair less invasive, resulting in faster recovery, versus repairs that use relatively large rulers, or guessing of the nerve size. The sizer measures by having about 180 degrees of contact with the root within the U shaped stump receptor 380. The fit of the nerve stump in the receptor can be done by feel, or visually. The advantage is that all the user needs to do is to decide if there are gaps or not in each receptor. There is a redundant check to see if the nerve is tangent to the reference face 390, which is a simple above or below decision. The tangency can also be done by feel with a finger. The sizer relies on checking the stump in two dimensions at once. It eliminates errors that may occur if the stump is inadvertently flattened during the measurement process, causing a too low or too high a reading. The sizer loosely holds the stump in a generally round section. The recesses are typically in one millimeter increments from 2 to 6 millimeters, but can be larger or smaller. The hub 370 can be held between the thumb and a finger, and the arms are quickly rotated to find the correct size, similar to how a fidget spinner toy is held and spun.

The scar trimmer 450 shown in FIGS. 23-32 allow for efficient stump trimming with one hand. The scarred or irregular shaped torn nerve end 580 will not heal as well as a freshly trimmed stump. The trimmer has a non-exposed blade, 500 or 510, with a cavity to fully support the stump to be trimmed 480. The cavity can be round or vee shaped 595 to allow the tool to be used on a range of size stumps. The cavity can be closed or open 595. The blade can be push activated, or reversed so the blade 1020 pulls to cut the stump. The blade has an angled blade so linear motion of the blade has an oblique shearing component, or can be straight perpendicular. The blade can have a non-serrated or serrated edge. The opening can have texture on it to prevent the nerve from moving during the cutting motion. The housings 455 and 460 can be screwed together or alternately use integral snap fit fasteners for more economical and faster assembly. The surfaces opposite the blade 490 will provide support on each side, a desirable double shear condition.

The activation button 470 is typically pushed with the thumb to advance the blade. There is a spring 530 that returns the blade to the retracted position. After the nerve is cut, the blade disappears into the housing. An industry standard blade 500 can be used in the trimmer, a number 10 blade is shown in the drawings. Custom blades 510 can be used for shorter stroke of operation, and to optimize cutting efficiency, largely driven by angle of the cutting edge. The trimmer allows cutting very thin section of scar and nerve, to expose fresh cells, while maintaining as much length of the stumps as possible.

The conduit carrier 600 is shown in FIGS. 33-55. Its main purpose is to hold a conduit and facilitates its placement. The carrier supports the conduit by capturing regions of the conduit in numerous places to maintain a cylindrical inner diameter. Preferably the tabs 130 from the exterior mate with recesses 870 between the carrier jaws 660. The jaws 660 can be a cylinder divided into between 2 and 6 segments.

The interior of the carrier is hollow 775 throughout its length. One on end, the carrier connected to a readily available suction source in an operating room by having a boss to slip onto. The hollow chamber 775 goes through the jaw part of the instrument, through the vacuum port of the wall of the conduit 100, and into the center of the conduit.

The carrier is placed so the stumps are close to the entry 150 of the conduit. When the stumps are properly positioned, a finger is placed over the port 650, and the pressure in the conduit drops. The narrow gap between the nerve stump diameter and the conduit restricts incoming air, and the vacuum, pressure differential, draws the stump into the center of the conduit, without the need to pull, push manually or use sutures. The other side is pulled in in the same manner. To prevent the first nerve from completely blocking the vacuum port thus rendering a second coaptation impossible, a separator 710 or 800 is placed where the stumps are ideally positioned.

During repositioning, continuous use or intermittent use of the vacuum port 650 may be helpful. A vacuum valve 1590 acts as a fixed port cover, can be used to keep the vacuum coaptation force engaged without the need for a finger. This frees the surgeon's hand to place sutures 890 between the epineurium and the conduit for stability of the construct.

After coaptation, if the depth indicating mark is 2 millimeters from the exposed end of the conduit, that indicates the stump's end is adjacent to the separator and properly positioned to heal best. If the mark is more than 2 millimeters from the end, there may be some restriction in the nerves ability to move into place, or the carrier is not in the ideal central location. Adjustment of the nerves and carrier will free the impediment, and allow proper coaptation.

The jaws 660 are held together with a radial compressive element 670, preferably a spring. An easy to access sliding button 640, distanced from the conduit, is used to open the jaws from pinching tightly on the tabs, to a disengaged position, while maintaining the jaws contact with the remainder of the instrument. The button 640 can snap back and forth, staying in the position desired, either closed or open.

The separator 710 can be integrated into the spring or button, so that as the conduit is being released, the separator is withdrawn, lower the risk that small hand movements will upset the repair.

The movement of the jaws releases the tabs 130, and lets the conduit be uncoupled from the carrier. The conduit is free to exit linearly from its previous position. The linear exit carrier only requires small incision.

The carrier fits in the hand like a pencil. A majority of the carrier's length is a tube 610, it can be metal or plastic, and can be assembled with a pin 700 or other hardware. The tube and housing could be made into one piece, or could be all plastic and be snapped together, maintaining a vacuum tight seal. The silicone sleeve 620 over a metal handle is optional for surgeon comfort and better grip of the instrument. Ergonomic sculpting of the sleeve can be incorporated.

There is some space 790 around the springs that will allow air to go into the vacuum chamber, slightly reduces the overall vacuum available for coaptation, but it does not reduce the ability to coapt significantly. The same is true of the gap between the stump and the conduit, a small gap 905 will not prevent coaptation.

FIGS. 40 through 44 show the operation of the spring 670 and separator 710. The jaws 660 have recess for maintaining a closed 750 and open 760 positions. The interlock between release button and spring is visible. The pinching of tabs between jaws is visible too. A wire separator 710 is integral to the spring, and fits through the vacuum port 110 in the conduit wall.

The central housings vacuum channel 775 is visible in the sectional view 45 where the spring shares the vacuum channel.

FIG. 46 is the conduit carrier central housing is the main gripping component, it has recesses 780 for the positioning of the mobile separator. It has a smooth exterior for ease of gripping, and has a cross section of between 8 to 25 mm wide and tall. It can be molded from polymer for light weight.

FIG. 47 is an alternate embodiment of a fixed separator. It would be coupled with a simplified version of spring 670.

The coaptation in-process is show in FIGS. 48-51. In FIG. 50, The nerve on the left is fully coapted to the separator, and the nerve on the right is partial coapted.

FIG. 51 shows the pinch points of the tabs between the support jaws. The tabs are shown in their neutral (uncompressed) position. The true position of the tabs would be deformed to fit the gaps in the jaws. The contact faces of conduit carrier central housing 630 are angled to avoid shearing the tabs. The included angle of the faces of the recessed 870 of conduit carrier central housing 630 is thirty degrees, and could be between zero and 90 degrees.

The jaw in detail is in FIGS. 52-55. There are two identical jaws 660 used in the carrier 600. The jaws mate the other carrier components at surfaces, 865. There are optional interlocking features 765 and 770, on the jaws at the 3 o'clock position of FIG. 52. This maintains rigidity of the instrument during handling and use. The distance from the conduit cylinder to the hinge is 9 millimeters, and can range from 5 to 200 millimeters. The jaw's partial cylinder boss, 765, will vary in diameter to support conduits of various sizes. The jaws are sized to work with a 4 mm diameter conduit. The length of the partial cylindrical bosses are typically three times the conduit diameter but can vary from 5 to 50 millimeters.

A completed direct repair with a conduit and two nerve stumps is shown in FIGS. 56-58. The coaptation is complete with the nerve stumps inside the conduit, with a regeneration gap 895 that is between 0.1 and 5 millimeters, with the ideal being about 1 millimeter, the width of a separator. The nerve stumps have nearly equal contact with the conduit. Sutures to hold the conduit in place are shown spaced around the edge 890. The vacuum port flaps 160 are shown in the closed position, being smooth on the inside and outside of the conduit.

The vacuum coaptation process has a smaller radial gap 905 than conventional drawn suture technique, which may require the conduit be oversized from 1 to 3 millimeters. This takes up less room in the wound, and is likely will result in less errant axon growth down the radial gap 905.

Coaptation force is calculated by multiplying the cross sectional area of the inside of the conduit by the differential between the pressure in the vacuum chamber and atmospheric pressure. Typical hospital operating room suction systems are operated at 150 millimeter of mercury. For each stump, the results are shown in the Table 1 below. These forces will readily move the stump in place, and do not overload the anatomy or damage the stumps.

TABLE 1

| Conduit | Coaptation |
|---|---|
| Diameter | Force |
| millimeters | Newtons |
| 1.5 | 0.04 |
| 2 | 0.06 |
| 3 | 0.15 |
| 4 | 0.26 |
| 5 | 0.4 |
| 6 | 0.58 |
| 7 | 0.79 |
| 8 | 1.04 |
| 9 | 1.31 |
| 10 | 1.62 |

A stepped diameter conduit 900 is shown in FIGS. 59-61. The windows 260 near either end increase the ability of that region to stretch to a larger diameter. The windows are away from the vacuum port 110, and do not fully hamper the ability to perform vacuum coaptation. The reduced diameter ends 920 may be smaller than the nominal size of the nerve stump and could be used to hold the conduit to the stump with less or no additional suturing. Care must be taken as not to compress the nerve too much with the ends, causing injury. The vacuum port is in the center, and works with a vacuum coaptation instrument. A set of vacuum port flaps 120 may be used with this embodiment, as can be external tabs to maintain shape. The end diameters are shown as sized between 0.5 to 4 millimeters less than the center section of conduit diameter, which ranges from 2 to 10 millimeters in diameter. Construction material would typically be a flexible silicone or a bioabsorbable fibrous weave.

The carrier for the rolled back conduit 930 is shown in FIGS. 62-65. The conduit's rolled ends 220 go outside of the two piece cylinder that is formed by the mating of the partial cylindrical plane of carrier for rolled end conduit 930 and the partial cylindrical surface of the floating jaw 950. The rolled back ends are wrapped around each end of the formed cylinder, providing complete radial support of the conduit 200. The unrolling tabs 240 are held out of the way on dedicated bosses, but are easily accessible for unrolling, held on by a boss 970 being slightly larger than the tab opening. The unrolling tabs are gripped by the large tab and moved away from the conduit, in line with the nerve. The tab pulls the unrolled end outward, and as it is pulled it unrolls the entire circumference. The tab has contact with about twenty percent of the rolled end, and could be between 5 and 50 percent. Both ends are unrolled, and both unrolling tabs are trimmed off. The conduit will hold the cylindrical components 950 and 990 together.

Optionally, the floating jaw 950 can be held onto the housing by a tensioned flexible retainer (high strength thread) 960, that is anchored away from the conduit, and is cut to release the jaw. The thread is attached to a spring 1860 to maintain tension on the jaw. The threads provide high force to hold the jaw stationary while being very low profile and having no need for extra components. The jaw can have interlocking features for axial stability. The stabilizer 940 can be on the floating jaw as pictured or can be on the housing side. A slidable separator could be fitted to this embodiment. Once the thread is cut, the housing is removed from the repair site, and the floating jaw is retrieved from the repair site.

An alternate embodiment of the conduit carrier is having jaws that have inwards facing bosses to pinch material between the windows to allow maintenance of the conduit diameter without external tabs on the conduit, 900. The release and operation of this embodiment is otherwise the same as a tabbed conduit.

The split contact face can alternately be aligned with the vacuum channel axis.

A completed long gap nerve repair construct 1000, is shown in FIGS. 66-68. For nerves that are more damaged, typical in crush injuries, a practitioner may elect to leave a gap of 20-30 millimeters. To accomplish this, the large gap conduit 1010 and matching carrier will be extended to a length between 15 and 50 millimeters long, with diameters between 1.5 and 10millimeters The vacuum assisted coaptation process is the same as the direct repair would be. The suturing of conduit to the nerve is done as it is in a direct repair. The large gap conduit may be silicone or bioabsorbable, and can have a wall thickness between 0.2 millimeters to 4 millimeters.

A pull cutting blade 1020 for the scar trimmer is shown in FIG. 69. This is an alternate embodiment of the push blade, instead of cutting on the push stroke, it cuts on the pull stroke. The pull blade is slightly harder to make than the external cutting edge, but may allow the trimmer to fit into smaller wounds.

The use of conduits 100 and a nerve graft 1220 in defects from 20 to 30 up to 70 millimeters is shown in FIGS. 73-75. In cases where the viable nerve gap is over two centimeters, the preferred method is to place a nerve graft between the stumps. This requires twice the effort on the surgeons' part. The procedure is streamlined by using the present invention, on each conduit connection to the nerve graft. The vacuum coaptation is done with a graft and stump. The repairs would likely be done sequentially. The graft to stump repair would function very much like a direct repair.

A carrier support stand 1300 is in FIGS. 76-77. It has a wide base for stability, as it functions as a tripod, the base having two spaced two contacts 1320, the third being the conduit carrier. The carrier support stand is engaged to the carrier by placing the vacuum connection cylinder (handpiece) into the carrier tube recess 1310 on the top. The feed channels snap over the mating part 610, preventing it from falling off. It can rotate about the carrier and can be moved closer or farther from the conduit to vary the holding angle. A light interference fit will hold a relative position, and can be easily slid for adjustment. The depth of the stand is between 3 and 15 millimeters. The base is between 25 and 75 millimeters wide, and has a height of between 25and 250 millimeters. It is preferably made from polycarbonate or another polymer. It can have a canted version of the carrier tube recess. Other connection means to the carrier could be utilized. The stand would replace a surgical assistant that otherwise would need to position the carrier during the anchoring process. The stand will be more stable than a human could hold the carrier, making anchoring easier.

Alternate embodiments of a wrap conduit, 1400 and 1450, are shown in FIGS. 78-79. Wrap conduits are another common way to get the stumps coapted. The vacuum coaptation of the carrier can be adapted to the wrap if the wrap 1400 has radial tabs 1420 spaced arounds its circumference, maintaining a generally cylindrical profile. There can be tabs at the inner end of the spiral to create a larger diameter than its resting diameter. Once the instrument releases the tabs, the wrap conduit goes back to its original size that it was created at. All tabs radial and those emanating from the ends, are captured in a similar instrument as the continuous wall conduit 100 utilizes. It has a similar vacuum port 100 and coaptation method. The wrap conduit has the ability to repair nerves of various sizes, making sizing less critical as the nominal inner diameter of wrap conduit can vary several millimeters. It may be better for nerves that have unusual shapes. This size range would allow the treatment of patients with less hospital inventory. The internal spiral end 1425 can have a chamfered corner to reduce impingement on the nerve. The internal spiral end 1425 will move between the 4 o'clock position and 8 o'clock position. The 4 o'clock position shown is when the inner diameter is at its largest size, for ease of nerve coaptation. The tabs can be shorter such as shown in retaining tabs 130 with the wrap conduit 100. A wrap conduit with the spiral having more revolutions than shown can be adapted to the carrier instrument.

A carrier for the wrap conduit with end tabs, 1450 can have a staged release, where the spiral end tabs 1565 are released first, allowing the inner diameter to partially closed, and a second release that will full release the conduit. This can make for better vacuum coaptation due to less radial gap, 905.

An alternate embodiment of the carrier is the lever carrier, 1500, shown in FIGS. 81-85 and 70-72. The lever jaws 1520 on the carrier pivot 12 millimeters from the conduit center axis, with a range of 8 to 200 millimeters. The alternate capture and release mechanism is done via a lever that holds and releases the conduit. A clothes pin style spring 1530 is used to provide the force to immobilize the jaws prior to implantation. The vacuum is controlled via finger operated vacuum port.

The carrier can have embodiments with a vacuum valve 1590 and both with and without a separator 800. The separator may not be required on large gap repairs.

If the central housing is nearly as wide as the conduit, the spacing of the internal vacuum channel could be very wide, and coaptation may be possible without a separator.

The jaws could be three pieces, with an internal vacuum channel and two mobile jaws, or could only have two mobile jaws if they had half of a vacuum central channel in each. The lever jaws can have a catch so the lever will remain open once it has been squeezed and remains oriented as shown in FIG. 85. A mobile separator is adaptable to the lever carrier instrument. FIG. 72 shows the two contact surfaces on the outer face, at the 3 and 9 o'clock positions. The carrier housing and tube could be molded as one piece.

An alternate embodiment of the conduit is in FIGS. 86-88. The tabs by the conduit ends are in a single plane, 180 degrees apart, and require only two, at 3 and 9 o'clock positions as shown in FIG. 87. This tab arrangement works with stiffer conduits. The tabs adjacent to the vacuum port are three in number, and are roughly equally spaced, at 3, 8 and 10 o'clock positions. The additional tab provides a good vacuum seal. This is the conduit used in the lever carrier in FIGS. 81-85. The conduit has a vacuum port, and can have flaps to close it after implantation. The conduit could have a separator in its center that is a part of the conduit material, and remains in the repair. The separator could be a small thread or thread matrix that would limit coaptation, but not impede cell growth. The separator could be a thread or suture that is tensioned during implantation, but removed prior to completing the repair.

The lever jaw embodiment of the carrier shows another embodiment to holding the conduit in the ready position. Where the first embodiment had three approximately equally spaced tabs 130 around the exterior of the conduit, the 180 degree jaw version has multiple zones of tab geometry. The conduit closest to the vacuum port, has three generally equal spaced tabs 1560, but at the outer edges, there are two tabs 1550. This configuration will work with conduits that have a wall that is stiff enough to support itself. The three tab version is better able to support thin and conduits less able to support themselves, especially if high vacuum is used. Three tabs by the conduit vacuum port will better seal the interior of the conduit to the carrier's vacuum channel. The jaws for mating with 1540 will have three contact planes, at the ends at 3 and 9 o'clock, and near the separator, it will have contact faces at 3, 8 and 10 o'clock positions (not shown)

A tensioned thread could be used to hold the hinged jaws together in place of a dedicated spring. The thread could be released by uncoupling its anchor loop instead of cutting.

The tensioned thread used with a floating jaw or lever jaw could be looped on a boss just after crossing the jaw contact plane, so that only a short amount of thread is required to be pulled past the repaired nerves instead of half the loop as shown in FIG. 64.

The carriers without separators and conduits with vacuum port flaps will work as the vacuum is high enough to pull the flaps open enough to coapt the nerves. This is how prosaic industrial flapper valves work.

An alternate embodiment of the conduit carrier is shown in FIGS. 89-92. The simplified device has no moving components. It has a tubular handpiece 1650 and separator 1670, a contact face 1720, and geometric interlocking material 1680. It is paired to a conduit that has a round vacuum port. The carrier is threaded into the vacuum port, or if the interlocking material is unthreaded, it can be pushed over the seat the conduit to the contact face. The interlocking material can be a part of the handle or another piece, which could be changed in effective cross sectional area by a remote activated lever, collet or similar means. The engagement or disengagement is done by retracting the handle, or unthreading it.

The separator 1670 contacts the opposite side of the interior channel, and provides a place to stop nerve stumps from being in direct contact with each other and retarding or preventing the development of the axonal growth cone. The separator has vacuum paths 1660 to allow air to move from either side of the conduit. The carrier connects to a vacuum line, and has an internal vacuum channel 1690. This embodiment is best suited to conduits made from materials and with wall thicknesses that are self-supporting, as the instrument does not have additional radial support.

A multiple vacuum port conduit 1800 is shown in FIG. 93. It has tabs 130 to maintain shape. It has multiple vacuum ports 110 to have direct vacuum coaptation forces independently. The multiple ports may not require a separator be used to properly position the stump, but requires the surgeon center the conduit prior to coaptation. It is mated to a carrier that is seen in FIG. 33 or 81 with a modification to the central housing. This approach may be better suited to large gap repairs where the conduit is longer than shown.

The conduit with linked tabs 1820 is shown in FIG. 94. The space created a capture recess 1850, can make loading of the conduit into a carrier easier. The linking segment can be trimmed off or left on at the surgeons' discretion. The radial tab segments 1830 are shown only near the ends of the conduit, but there could be between 1 and 8 tabs along the length of the conduit.

The surgical method disclosed herein includes the step of trimming the scar. To trim scar, slide trimmer 450 over the end of the nerve stump. Push activation button 470 to slice off damaged tissue. If viable cells are not visible across the face of the stump, repeat the cut. The trimmer allows very thin slices to be made. Repeat on the other stump.

Using a surgical marker, another step is to make a mark on the nerve stump approximately 7 millimeters from the end just trimmed. Repeat on the other stump. This will be used to confirm proper coaptation later in the technique.

To measure the nerve root diameter, put the nerve into U recess on the end of the arm of the sizer 350. Proper fit means the nerve will fit in and be tangent at the upper surface.

The selected conduit will be slightly larger than the nerve, about 0.5 millimeters.

Another step is to connect the conduit carrier 600 to the operating rooms vacuum supply with a suction line.

If the conduit is bioabsorbable, place in saline to wet the material.

Another step is to position conduit carrier between the stumps.

Another step is to move the carrier very close to one stump.

Another step is to close the vacuum control port 650 with a finger, the nerve stump will advance into the captured conduit with folded tabs 170. For additional coaptation force, put another finger on the other side of the conduit.

Another step is to move the conduit carrier closer to the second nerve stump, closing the vacuum control port will cause the second nerve to go into the conduit. The conduit carrier has a separator 710 that positions the nerve ends in the proper position for regeneration. A mobile retractor 710 will not stress the repair much during carrier removal. Fixed separator 800 will properly orient the nerve ends for regrowth.

It should be noted that simultaneous coaptation of both nerves is possible depending on anatomy.

In addition, suture conduit to epineurium if desired. Release the carrier from the conduit and gently retract the carrier from the wound.

Alternative or option steps in the surgical method may include the carrier's vacuum valve being engaged to hold the stumps inside the conduit, without the need to keep a finger over the port as keeping the port closed may keep the stump in the optimal coaptation position.

The carrier stand 1300 is snapped onto the tube of the carrier, and with its wide base will act as a tripod, and hold the carrier in place without the need for a surgical assistant to continually grip the part. The small motions of the assistant's hand may make suturing more difficult, which can take several minutes to complete. The carrier and carrier stand can be flipped over to the other side of the hand, allowing placement of sutures in other parts of the conduit.

Alternatively, the conduit can be used to connect the stump to one end of a graft, and another conduit to the other end of the graft.

### Exemplary Embodiments of the Invention

The following examples describe various exemplary embodiments and combinations of aspects of the invention described herein and illustrated in FIGS. 5-100. More specifically, the following provides examples of conduits, conduit carriers, nerve sizing instruments, scar trimmers and methods of performing a nerve repair procedure utilizing the same, as described herein and illustrated in FIGS. 5-100.

### Examples of Conduits for a Nerve Repair Procedure

A conduit 100 (FIG. 5), 170 (FIG. 9), 200 (FIG. 12), 900 (FIG. 59) 1400 (FIG. 79), 1540 (FIG. 86), 1695 (FIG. 91), 1800 (FIG. 93) 1810 (FIG. 94), 1875 (FIG. 95), 1885 (FIG. 96), 1900 (FIG. 97), 1960 (FIG. 100) in accordance with one or more aspects of the present disclosure, includes a conduit body including opposing first and second open end portions 150 and a hollow conduit interior extending therebetween. The conduit interior is configured to receive a first nerve stump 410 (FIG. 50) through the first open end portion 150 and a second nerve stump 410 through the second open end portion 150, during a nerve repair procedure on a patient.

The first and second nerve stumps 410 may include a combination of a nerve stump 410 inserted through one of the first or second open end portions 150 of the conduit 100 and a nerve allograft 1220 (FIGS. 73-75) inserted into the other of the first and second open end portions 150 of the conduit 100. The nerve allograft (or nerve graft) 1220 may be used to link two nerve stumps 410 that are spaced too far apart for a single conduit 100 to connect. For example, the combination of two conduits 100 and a nerve allograft 1220 may be used to link two nerve stumps 410 that are up to 70 millimeters apart.

The first and second nerve stumps 410 may include a proximal nerve stump and a distal nerve stump. The proximal nerve stump is positioned closer to the skeleton relative to the distal nerve stump. Nerve cells, such as axon nerve cells, generally grow from the proximal nerve stump. The distal nerve stump generally receives the out growing nerve cells from the proximal nerve stump.

A vacuum port 110 (FIG. 5) extends through an outer wall 140 of the conduit body. The vacuum port 110 is configured to connect to a vacuum source (not shown) associated with a conduit carrier 600 (FIG. 33), 1500 (FIG. 83) to pull a vacuum on the conduit interior while inserting the nerve stumps 410 into the first and second open end portions 150.

At least one radial support structure 130 (FIG. 5), 220 (FIG. 12) is positioned on the outer wall 140 of the conduit body. The at least one radial support structure 130, 220 is configured to structurally support the conduit body when the vacuum is being applied.

The at least one radial support structure of the conduit may be any support structure configured to structurally support the conduit body when the vacuum is being applied. However, several specific embodiments of the at least one radial support structure are discussed and illustrated throughout the present specification and drawings.

During a nerve repair procedure, the vacuum is used to create a low pressure within the interior of the conduit 100 in order to draw the first and second nerve stumps 410 into the first and second open end portions 150 of the conduit. The conduit 100 is often composed of a flexible material to allow for added patient comfort and bending of the patient's body part in which the conduit is implanted. Problematically, when the vacuum is applied, the flexible body of the conduit 100 has a tendency to collapse before the nerve stumps 410 can be inserted.

Advantageously however, the at least one radial support structures, such as one or more retaining tabs 130 or a pair of rolled back ends 220, may be used to provide structural support to the conduit 100. The radial support structures 130, 220 may also be designed to engage with the conduit carrier 600, 1500 to further maintain the shape of the conduit when the vacuum is being applied to the interior of the conduit.

In some embodiments of the conduit, at least one vacuum port flap 120 (FIG. 5) is configured to close over the vacuum port 110 when the vacuum is not being applied. More specifically, the vacuum port flap 120 can close over the vacuum port 110 when the nerve repair procedure is complete. When the vacuum port flap 120 is closed over the vacuum port 110, the vacuum port flap 120 prevents nerve cells from the first and second nerve stumps 410 from growing through the vacuum port 110.

There may be a plurality of vacuum port flaps 120 that are configured to close over a single vacuum port 110. For example, as illustrated in FIGS. 5 and 6, there may be a first and a second vacuum port flap 120 disposed on opposing sides of the vacuum port 110. The first and second vacuum port flaps 120 may close over the vacuum port flap 120 and abut together in the center of the vacuum port 110.

In some examples of the conduit 1800 (FIG. 93), the vacuum port 110 may include a first and a second vacuum port 110 (FIG. 93) that are spaced apart a predetermined distance. As will be explained in greater detail herein, the spaced apart vacuum ports 110 are used to accommodate larger regeneration gaps 985 (FIG. 68) between the first and second nerve stumps 410.

In some examples of the conduit, the at least one radial support structure 130, 220 includes at least one retaining tab 130 positioned on the outer wall 140 of the conduit body. The at least one retaining tab 130 is configured to enable jaws 660 (FIGS. 33-37), 1520 (FIGS. 81-85) of the conduit carrier 600, 1500 to engage therewith. For example, the jaws 660, 1520 may clamp onto the retaining tab 130.

In some examples the at least one retaining tab 130 includes a first set of retaining tabs 130 being positioned proximate the first open end portion 150 and a second set of retaining tabs 130 being positioned proximate the second open end portion 150 of the conduit 100 (see FIGS. 5-8). The vacuum port 110 is positioned centrally on the conduit body between the first and second sets of retaining tabs 150. In some examples of the conduit, the first set of retaining tabs 130 may include three tabs 130 spaced apart between 60 and 180 degrees on the outer wall 140 of the conduit body (see left side of conduit 100 in FIG. 5). Additionally, the second set of retaining tabs may include three tabs 130 spaced apart between 60 and 180 degrees on the outer wall 140 of the conduit body.

In some examples of the conduit, at least one retaining tab opening 190 (FIG. 9) is disposed on the outer wall 140 adjacent the at least one retaining tab 180 (FIG. 9). The at least one retaining tab 180 is foldable into the at least one retaining tab opening 190 (FIG. 10).

In some examples of the conduit, the at least one retaining tab 180 includes a plurality of retaining tabs 180 and the at least one retaining tab opening 190 includes a plurality of retaining tab openings 190. Each retaining tab 180 of the plurality of retaining tabs 180 is foldable into an associated adjacent retaining tab opening 190 of the plurality of retaining tab openings 190.

In some examples of the conduit 1400 (FIGS. 78-80), the at least one retaining tab includes a continuous support tab 1420 extending longitudinally along a length of the conduit body from the first open end portion 150 to the second open end portion 150. As will be explained in greater detail herein, these continuous support tabs 1420 may be disposed on a conduit 1400 having a spiral wall 1410.

In some examples of the conduit 1810, the at least one retaining tab includes a linked tab 1820. The linked tab 1820 includes a first and a second radial tab segment 1830 extending radially outward from the outer wall 140. Additionally, the linked tab 1820 includes a linking tab segment 1840 integrally connected to the first and second radial tab segments 1830 and extending longitudinally therebetween. The linked tab 1820 and outer wall 140 define an open capture recess 1850 therebetween. The capture recess 1850, can make loading of the conduit 1810 into a conduit carrier 600, 1500 easier. The linking tab segment 1840 may be trimmed off or left on at the surgeons' discretion.

In some examples of the conduit 1900 (FIG. 99), the at least one retaining tab includes a dovetail tab 1910. The dovetail tab 1910 includes a lower tab end (bottom of the tab 1910) and an upper tab end (top of tab 1910). The lower tab end is attached to the outer wall 140 and has a first width. The upper tab end has a second width. The second width is larger than the first width to provide the dovetail shape.

In some examples of the conduit 1960 (FIG. 100), the at least one retaining tab includes a stepped tab 1970. The stepped tab 1970 includes a lower tab segment and an upper tab segment. The lower tab segment is attached to the outer wall 140 and has a first width. The upper tab segment is disposed over the lower tab segment. The upper tab segment has a second width. The second width is larger than the first width to provide the stepped shape.

In some examples of the conduit 1400 (FIGS. 78-80), the conduit body includes a spiral wall 1410. The spiral wall 1410 includes a longitudinally extending first wall end portion and a longitudinally extending second wall end portion. The first wall end portion overlaps and abuts against the second wall end portion (see FIG. 78) to form the hollow conduit interior.

In some examples of the conduit, the conduit includes a stepped diameter conduit 900 (FIGS. 59-61). The stepped diameter conduit 900 includes a first open end portion 920 having a first internal diameter. The conduit 900 also includes a second open end portion 920 having a second internal diameter. A central section 910 of the conduit body is disposed between the first and second end portions 920. The central section 910 has a third internal diameter. In the stepped diameter conduit 900, the first and second internal diameters are smaller than the third internal diameter. In the example of FIGS. 59-61, the first and second internal diameters of the first and second open end portions 920 are substantially equal.

The stepped diameter conduit 900 may also include a plurality of first elasticity tuning openings 260 disposed in the first open end portion 920 (left side of conduit 900). The first elasticity tuning openings 260 are configured to provide a predetermined elasticity to the first open end portion 920 (left side of conduit 900). The conduit 900 may also include a plurality of second elasticity tuning openings 260 disposed in the second open end portion 920 (right side of conduit 900). The second elasticity tuning openings 260 are configured to provide a predetermined elasticity to the second open end portion (right side of conduit 900). The predetermined elasticity may be used to secure the conduit 900 to the nerve stumps 410 without the need for additional suturing.

The first and second elasticity openings 260 can go at least partially through the wall of the conduit 900. The openings 260 can have various shapes, but rectangular are shown in FIG. 61. The removal of material makes the reduced diameter end 920 region more flexible than a stepped conduit 900 without openings 260. The opening parameters vary to allow the conduit 900 to lightly hold onto the nerve stump 410, over a wider diameter range than a conduit 900 without openings 260.

In some examples of the conduit 200, the conduit includes a rolled end conduit 200 (FIGS. 12-16). In the rolled end conduit 200, the at least one radial support structure includes the first open end portion 920 being configured to be rolled back upon itself 220 from a first extended position (FIG. 15, left side) to a first rolled back position (FIG. 12, left side). In the first rolled back position the first open end portion 290 structurally supports the conduit body when the vacuum is being applied and forms a first nerve entry region 230 (FIG. 12, left side) configured to receive the first nerve stump 410.

The at least one radial support structure also includes the second open end portion 920 being configured to be rolled back upon itself 220 from a second extended position (FIG. 15, right side) to a second rolled back position (FIG. 12, right side). In the second rolled back position the second open end portion 290 structurally supports the conduit body when the vacuum is being applied and forms a second nerve entry region 230 (FIG. 12, right side) configured to receive the second nerve stump 410.

The rolled end conduit 200 includes a first unrolling tab 240 detachably connected to a first distal end of the first open end portion 290 (left side of FIG. 15). The first unrolling tab 240 is configured to provide leverage to roll back the first open end portion 290 from the first extended position (FIG. 15) to the first rolled back position (FIG. 12). The rolled end conduit 200 also includes a second unrolling tab 240 detachably connected to a second distal end of the second open end portion 290 (right side of FIG. 15). The second unrolling tab 240 is configured to provide leverage to roll back the second open end portion 290 from the second extended position (FIG. 15) to the second rolled back position (FIG. 12).

The rolled end conduit 200 may also include a plurality of first elasticity tuning openings 260 disposed in the first open end portion 290. The first elasticity tuning openings 260 are configured to provide a predetermined elasticity to the first open end portion 290. The rolled end conduit 200 may also include a plurality of second elasticity tuning openings 260 disposed in the second open end portion 290. The second elasticity tuning openings 260 are configured to provide a predetermined elasticity to the second open end portion 290. The predetermined elasticity may be used to secure the conduit 200 to the nerve stumps 410 without the need for additional suturing.

### Examples of Conduit Carriers for a Nerve Repair Procedure

A conduit carrier 600 (FIG. 33), 840 (FIG. 48), 930 (FIG. 62), 1500 (FIG. 83), 1650 (FIG. 89), 1700 (FIG. 71) in accordance with one or more aspects of the present disclosure includes a carrier housing 630 (FIG. 33), 1510 (FIG. 83), 1655 (FIG. 89), 1710 (FIG. 71) comprising first and second contact ends and a vacuum channel 775 (FIGS. 45, 70 and 83), 1690 (FIG. 91) extending therebetween. The vacuum channel 775 is configured to connect to a vacuum source through the first contact end. An engagement mechanism 660 (FIG. 33), 1520 (FIG. 83), 1680 (FIG. 92), 1520 (FIG. 72) is connected to the second contact end. The engagement mechanism 660, 1520, 1680, 1520 is configured to releasably engage a conduit 170 (FIG. 33), 1540 (FIG. 83), 1695 (FIG. 92), 1540 (FIG. 72) during a nerve repair procedure on a patient.

The engagement mechanism of the conduit carrier may be any mechanism configured to releasably engage a conduit. However, several specific embodiments of the engagement mechanism are discussed and illustrated throughout the present specification and drawings.

The conduit carrier 600, 1500, 1650, 1700 also advantageously includes a separator 710 (FIG. 40), 1510 (right side distal end of housing 1510 in FIG. 83), 1670 (FIG. 92), 800 (FIG. 70). The separator is configured to extend from the second contact end. When the engagement mechanism 660, 1520, 1680, 1520 is engaged with the conduit 170, 1540, 1695, 1540 during the nerve repair procedure:
i. the vacuum channel 775, 1690 is connected through the second contact end to a vacuum port of the conduit 170, 1540, 1695, 1540 to enable the vacuum source to pull a vacuum 850 (FIG. 50) on an interior of the conduit, and
ii. the separator 710, 1510 (distal end thereof), 1670, 800 extends into the interior of the conduit 170, 1540, 1695, 1540 through the vacuum port 110 to provide a predetermined regeneration gap 895 (FIG. 58) between a first and a second nerve stump 410 inserted into the conduit.

The separator 710, 800 advantageously provides a positive stop that the nerve stumps 410 may abut against, (See FIG. 50, wherein nerve stump 410 on the left is in contact with the separator 710, and low pressure region 850 is still open.) This enables a more precise formation of the regeneration gap 895 than previous prior art systems and methods.

In some examples of the conduit carrier 600, 1500, 1700 the engagement mechanism includes a first and a second jaw 660 (FIG. 33), 1520 (FIGS. 71 83) connected to the carrier housing 630, 1510, 1710. The first and second jaws 660, 1520 are movable between a jaw closed position (FIG. 33, FIG. 71, FIG. 81) and a jaw open position (FIG. 35, FIG. 85). When in the jaw closed position, the first and second jaws 660, 1520 are configured to engage the conduit 170 (FIG. 34), 1540 (FIGS. 72 and 82) and the separator 710 (FIG. 41), 1510 (FIG. 83, right distal end thereof), 800 (FIG. 70) extends between the first and second jaws 660, 1520 into the interior of the conduit 170, 1540. When in the jaw open position, the first and second jaws 660, 1520 are configured to release the conduit 170, 1540 (see FIG. 36)

As will be explained in more detail herein, in the example of conduit carrier 600, the separator is movable from an extended first position (FIG. 40) to a retracted or withdrawn second position (FIG. 44). In the first position, the jaws 660 are closed around the conduit 170 and the separator 710 extends through the vacuum port 110 into the interior of the 170. In the second position, the jaws 660 are open and the separator 710 is withdrawn from the conduit 170.

By contrast, in the example of conduit carriers 1500 and 1700, the separator 800 is an integral part of the carrier housing 1510, 1710 and has a fixed position relative to the housing 1510, 1710. More specifically, the separator 800 is the extended right distal end of the carrier housing 1510, 1710 (i.e., the contact end of the housing 1510, 1710 which engages the conduit 1540).

In some examples of the conduit carrier 600, 1500, the first and second jaws 660, 1520 are movable laterally relative to the carrier housing 630, 1510, 1710 between the jaw closed position (FIGS. 40, 81) and the jaw open position (FIGS. 44, 85). In other words, the jaws 660, 1520 move sideways from the sides of the carrier housing 630, 1510, 1710. The lateral movement of the jaws 660, 1520 may be rotationally as well. That is, the jaws 660, 1520 may pivot at one end to move both rotationally and laterally relative to the carrier housing 630, 1510, 1710.

Advantageously, when the jaws 660, 1520 move from the jaw closed position to the jaw open position, the first and second jaws 660, 1520 are configured not to extend further longitudinally into an interior of the patient in order to release the conduit 170, 1540. In other words, unlike previous prior art conduit carriers (see FIGS. 2 and 3), the jaws 660, 1520 of the present invention are not required to penetrate further into the interior of a patient's body in order to be released. The primarily lateral movement of the jaws 660, 1520 relative to the conduit housing 630, 1510 reduces the risk of injury to the patient during the nerve repair procedure.

In some examples of the conduit carrier 600 the second contact end of the carrier housing 630 (i.e., the end of the carrier housing 630 that engages the conduit 170) includes a conduit contact face 810 (FIG. 47). The conduit contact face 810 is configured to conform to a shape of the conduit 170. Additionally, the separator 710 is configured to extend through the conduit contact face 810. The conduit contact face 810 and the first and second jaws 660 substantially surround the conduit 170 when the first and second jaws 660 are in the closed position (FIG. 40).

In some examples of the conduit carrier 1500, 1700, the carrier housing 1510, 1710 includes a first and a second jaw pivot boss 1570 disposed on opposing sides of the carrier housing 1510, 1710. The first and second jaw pivot bosses 1570 are configured to engage with the first and second jaws 1520 respectively to provide a fulcrum about which the first and second jaws 1520 can pivot between their respective jaw closed position and jaw open position. A lever spring 1530 (FIG. 83) is disposed in the carrier housing 1510, 1710. The lever spring 1530 is configured to engage with the first and second jaws 1520 to urge the first and second jaws to pivot into their respective jaw closed positions.

In some examples of the conduit carrier 600, 1500, 1700, when the jaws 660, 1520 in the closed position, the first and second jaws engage at least one radial support structure 130 of the conduit 1540 in order to structurally support the conduit 1540 when the vacuum is being applied. In some examples of the conduit carrier, the at least one radial support structure comprises at least one retainer tab 130 positioned on the outer wall 140 of the conduit body. Advantageously, the combination of the conduit carrier engaging with the radial support structure of the conduit helps to prevent the conduit from collapsing when a vacuum is pulled on the interior of the conduit.

In some examples of the conduit carrier 600, the first jaw 660 includes a first jaw contact face 865 (FIGS. 52-55) disposed on a distal end of the first jaw 660. The first jaw 660 also includes a first tab capture recess 870 disposed on the first jaw contact face 865. The second jaw 660 includes a second jaw contact face 865 disposed on a distal end of the second jaw 660. The second jaw also includes a second tab capture recess 870 disposed on the second jaw contact face 660. When the first and second jaws 660 are in the closed position, the first and second jaw contact faces 865 abut one another and the first and second tab capture recesses 870 surround and capture the at least one retainer tab 130. By surrounding the retainer tab 130 within the tab capture recess 870, the conduit carrier 600 more securely maintains the shape and position of the conduit 170 during a nerve repair procedure relative to prior art systems and methods.

FIGS. 97-100 show alternate profiles of the radial tabs, The dovetail tab 1910 has a larger width at its distal end and a smaller width at the conduit wall, wherein the transition between the larger width and the smaller width is a straight taper. The stepped tab 1970 has a stepped profile tab, with two distinct widths of the tab, the farther away from the wall being greater in width. Both of these tabs 1910, 1970 will interlock into the tab capture recess 870. Therefore, rather than relying on a friction fit to the carrier, there is a variation in compression or interference fit, that will not allow the tab 1910, 1970 to slide inwardly during coaptation, thus preventing collapse.

In some examples of the conduit carrier 600, a jaw spring 670 (or separator and jaw spring 670) having a first spring position (FIG. 40) and a second spring position (FIG. 44) is positioned within the vacuum channel 775 (FIG. 45) of the carrier housing 630. The jaw spring 670 includes a first and a second cantilever beam spring 720 (FIGS. 40, 44, 45) on opposing sides of the jaw spring 670. The jaw spring 670 also includes the separator 710 positioned between the first and second cantilever beam springs 720. When in the first spring position (FIG. 40), the first and second cantilever beam springs 720 urge the first and second jaws 660 into the jaw closing position respectively, and the separator 710 extends into the interior of the conduit 170. When in the second spring position (FIG. 44), the first and second cantilever beam springs 720 urge the first and second jaws 660 into the jaw opening position respectively, and the separator 710 is withdrawn from the interior of the conduit 170.

In some examples of the conduit carrier 1650 (FIGS. 89-92), the engagement mechanism comprises the second contact end having a threaded portion 1680 (FIG. 92) through which the separator 1670 extends. The threaded portion 1680 is configured to thread into the vacuum port 110 of the conduit 1695.

In some examples of the conduit carrier, the separator 710, 800 has a separator width of between 0.1 and 5 millimeters and the predetermined regeneration gap 895 is substantially equal to the separator width. Advantageously, the separator 710, 800 provides a positive stop for the nerve stumps 410 when being inserted into a conduit 100, therefore providing more precise control of the regeneration gap 895 than previous prior art systems and methods.

In some examples of the conduit carrier 1700, the separator 800 may include a first and a second separator 800 extending in parallel (not shown) from the end of the conduit carrier 1700. The first and second separators 800 have first and second separator widths respectively and are spaced apart a predetermined distance. The first and second separators 800 are configured to extend through a first and a second vacuum port 110 (FIG. 93) of the conduit 1800 respectively. Accordingly, the predetermined regeneration gap 895 is substantially equal to the predetermined distance between the first and second separators 800 plus the first and second separator widths. The two parallel separators 800 provide a positive stop of nerve stumps 410 that have a regeneration gap 895 that is larger than the width of a single separator 800. For example, the regeneration gap 895 may be between 5 and 10 millimeters.

In some examples of the conduit carrier 930 (FIGS. 62-65), the conduit carrier 930 may be a conduit carrier 930 for a rolled end conduit 200 (FIGS. 12-16). The attachment mechanism of the conduit carrier 930 may include a partially cylindrical surface 990, a floating jaw 950 and a flexible retainer 960.

The partially cylindrical surface 990 is positioned at the second contact end (i.e., the contact end which engages the conduit 200) of the conduit carrier 930. The partially cylindrical surface 990 is configured to fit over a first portion of an outside surface of the rolled end conduit 200 when the conduit 200 is mounted on the partially cylindrical surface 990.

The floating jaw 950 is configured to fit over a second portion of the outside surface of the rolled end conduit 200 when the rolled end conduit is mounted on the partially cylindrical surface 990. The partially cylindrical surface 990 and the floating jaw 950 are configured to substantially surround the rolled end conduit 200 when it is mounted on the conduit carrier 930.

A separator 940 may extend longitudinally from one of the partially cylindrical surface 990 or the floating jaw 950. That is, the separator 940 may protrude from the either the partially cylindrical surface 990 of the body of the conduit carrier 930, or alternatively, may protrude from the concave surface of the floating jaw 950.

The flexible retainer 960 is attached to, and spring loaded by, a retainer spring 1860 disposed within the carrier housing of the conduit carrier 930. The flexible retainer 960 is configured to loop over the floating jaw 950 to elastically secure the rolled end conduit 200 between the floating jaw 950 and the partially cylindrical surface 990. The combination of the floating jaw 950 and partially cylindrical surface 990 substantially surrounds the rolled end conduit 200.

Additionally, the carrier housing of conduit carrier 930 may include a first and a second unrolling tab holding boss 970 disposed on opposing sides of the carrier housing. The first and second unrolling tab holding bosses 970 are configured to removably secure the first and the second unrolling tabs 240 of the rolled end conduit 200, when the rolled end conduit 200 is mounted on the conduit carrier 930.

As described herein with reference to FIGS. 12-16, the rolled end conduit 200 includes the first and second open end portions 290, which are configured to be rolled back upon themselves from a first extended position (FIG. 15) to a first rolled back position (FIG. 12). When the rolled end conduit 200 is mounted on the conduit carrier 930 and the first and second open end portions 290 are in the first and second rolled back positions (FIG. 12, FIG. 65) respectively, the first and second open end portions 290 are rolled over opposing lateral ends of the partially cylindrical surface 990 and floating jaw 950 (FIG. 65) to form the rolled back ends 220 as illustrated in FIG. 65. Accordingly, the combined lateral ends of the partially cylindrical surface 990 and the floating jaw 950 provide 360 degrees of structural support (FIG. 65) for the rolled end conduit 200 during a nerve repair procedure.

In some examples of the conduit carrier 600, the conduit carrier includes a vacuum control port 650 disposed on the carrier housing 630. The vacuum control port 650 extends through the carrier housing 630 to the vacuum channel 775. When the vacuum source is pulling a vacuum in the interior of the conduit 170 through the vacuum channel 775, a pressure of the vacuum in the interior of the conduit 170 is controlled when a finger of an operator (not shown) is placed over the vacuum control port 650. Accordingly, the pressure of the vacuum may be controlled by hand during a nerve repair procedure. The port can be omitted for continuous coaptation without the need to hold a finger on the carrier.

In some examples of a conduit carrier, the conduit carrier 1500 may include a carrier stand 1300 (FIGS. 76, 77) for supporting the conduit carrier 1500 during the nerve repair procedure. The carrier stand 1300 includes a carrier base 1320 (or carrier stand base 1320) having at least a first and a second leg for supporting the carrier stand 1300 in an upright position. A stem extends in a vertically upward direction from the carrier base 1320. A carrier housing receiving section 1310, 1330 is disposed on an upper distal end of the stem. The carrier housing receiving section 1310, 1330 includes a resilient carrier tube feed channel 1330 and a carrier tube recess 1310. The resilient carrier tube feed channel 1330 is configured to frictionally allow a portion of the carrier housing 1510 to pass therethrough. The carrier tube recess 1310 is integrally connected to the carrier tube feed channel 1330. The carrier tube recess 1310 is configured to receive and secure the portion of the carrier housing 1510 passed through the carrier tube feed channel 1330.

### Examples of Nerve Sizing Instruments for a Nerve Repair Procedure

A nerve sizing instrument 350 (FIGS. 17-22) in accordance with one or more aspects of the present disclosure includes a plurality of U-shaped recesses 380. Respective recesses 380 of the plurality of U-shaped recesses 380 are configured to receive therein a nerve stump 410 having up to a predetermined nerve stump maximum diameter that is different from any other nerve stump maximum diameter associated with any other respective recess 380. In other words, the U-shaped recesses 380 of the nerve sizing instrument 350 may be sized to receive a nerve stump 410 having a predetermined maximum diameter, wherein no two recesses 380 are sized to receive the same nerve stump maximum diameter.

In some examples of the nerve sizing instrument 350 each and every U-shaped recess 380 of the plurality of recesses is sized for a different nerve stump maximum diameter. In some examples of the nerve sizing instrument 350, each respective U-shaped recess 380 has a recess depth and a recess width that are equal to the nerve stump maximum diameter associated with the respective recess 380.

Advantageously, with the depth and width of the U-shaped recess 380 being equal to the maximum diameter of the nerve stump 410 that the recess is sized for, then the top of the best fit nerve stump 410 into the recess 380 will be tangent to the top surface of the recess 380 (see FIG. 22). Therefore, a surgeon can determine the diameter of the properly fitted stump 410 by simply feeling by hand that the top of the nerve stump is level with the top of the recess 380. The surgeon does not have to actually see the nerve stump to determine its size with the present nerve sizing instrument 350.

In some examples of the nerve sizing instrument 350, the nerve stump maximum diameter associated with each respective U-shaped recess 380 is incremented by 1 millimeter relative to an adjacent respective recess 380.

In some examples of the nerve sizing instrument 350, the nerve sizing instrument includes a central hub 370. A plurality of arms 360 extend radially from the hub 370. The respective recesses 380 of the plurality of U-shaped recesses 380 are disposed on distal ends of respective arms 360 of the plurality of arms 360.

### Examples of Scar Trimmers for a Nerve Repair Procedure

A scar trimmer 450 (FIGS. 23-29 and 69) in accordance with one or more aspects of the present disclosure includes a longitudinally extending housing assembly 455, 460 having a forward end (right end in FIG. 23) and a rearward end (left end in FIG. 23). The forward end has a forward boss 490. The forward boss 490 includes a stump cutting cavity 480 disposed therein. The stump cutting cavity 480 is configured to receive a nerve stump 410 extended therethrough. An internal slider 560 is disposed within an interior of the housing assembly 455, 460. The internal slider 560 is movable between a forward position and a rearward position relative to the housing assembly 455, 460. A blade 500 is slidably captured by the internal slider 560. When a nerve stump 410 is extended through the stump cutting cavity 480 and the internal slider 560 is moved between the rearward position and the forward position, the blade 500 is slide through the stump cutting cavity 480 to trim a scarred end of the nerve stump 410 off.

In some examples of the scar trimmer 450, the internal slider 560 also includes an activation button 470. The activation button 470 protrudes through the housing assembly 455, 460 and is slidably disposed thereon. The activation button 470 is configured to move the internal slider 560 between the rearward position and the forward position by hand in order to trim the scarred end of the nerve stump 410 off.

In some examples of the scar trimmer 450, the internal slider 560 also includes a blade capture boss 540 disposed forward of the activation button 470. The blade capture boss 540 slidably captures the blade 500. The internal slider 560 also includes a spring capture loop 535, which is disposed rearward of the activation button 470. A spring 530 is disposed within the spring capture loop 535. When the internal slider 560 is moved from the rearward position to the forward position by hand, the blade 500 is slide forward through the stump cutting cavity 480 to trim the scarred end of the nerve stump 410 off. When the activation button 470 is released by hand, the spring 530 urges the internal slider 560 from the forward position to the rearward position to slide the blade 500 rearwardly out of the stump cutting cavity 480.

In some examples of the scar trimmer 450, the blade 1020 (FIG. 69) is a pull cut blade 1020, which is captured by the internal slider 560. When the internal slider 560 is moved from the forward position to the rearward position, the blade 1020 is slide rearward through the stump cutting cavity 480 to trim the scarred end of the nerve stump 410 off.

In some examples of the scar trimmer 450, the stump cutting cavity 480 includes an open stump cutting recess (or vee recess) 595 integrally connected to a stump cutting access slot 590 (FIG. 30). The stump cutting access slot 590 is configured to hook over a nerve stump 410 in order to position the nerve stump 410 within the stump cutting recess 595 to be trimmed by the blade 500, 1020.

The scar trimmer housing assembly 455, 460 may include a scar trimmer housing side 450 and a scar trimmer housing notched side 460, which function as contact regions for the nerve stump 410 as the nerve stump 410 is extended through either the closed or open embodiments of the stump cutting cavity 480. The blade 500 may move between the scar trimmer housing side 450 and the scar trimmer notched side 460 of the housing assembly 455, 460 as the blade slides forward or rearward through the stump cutting cavity 480. Accordingly, the blade 500 advancing forces go into cutting, not making the nerve stump 410 slide out of position.

### Examples of Methods of Performing a Nerve Repair Procedure On a Patient

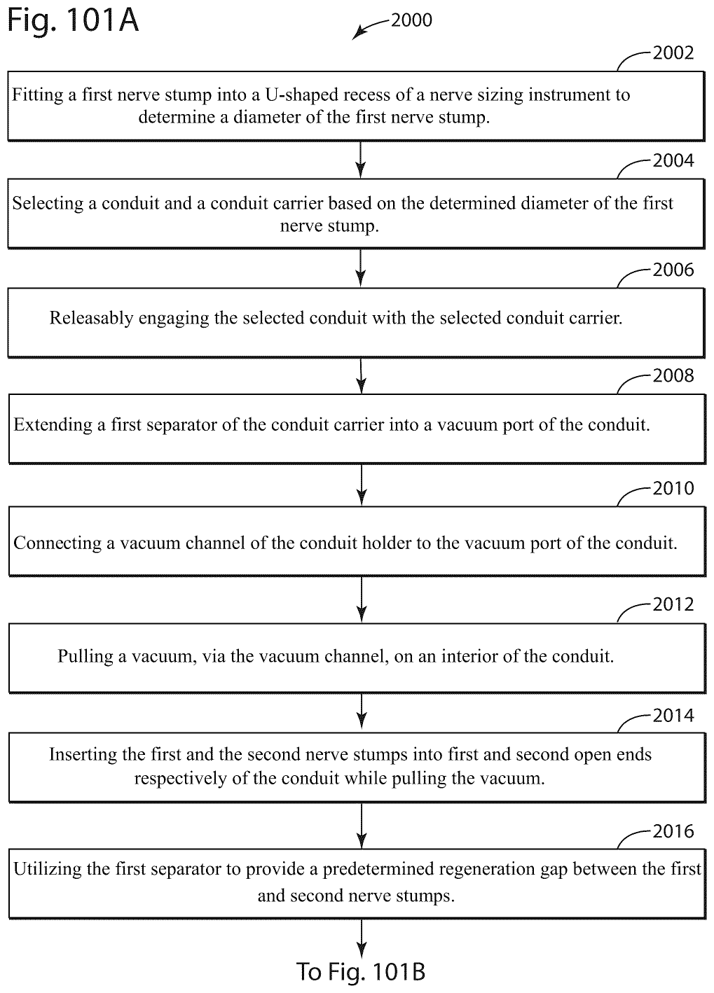

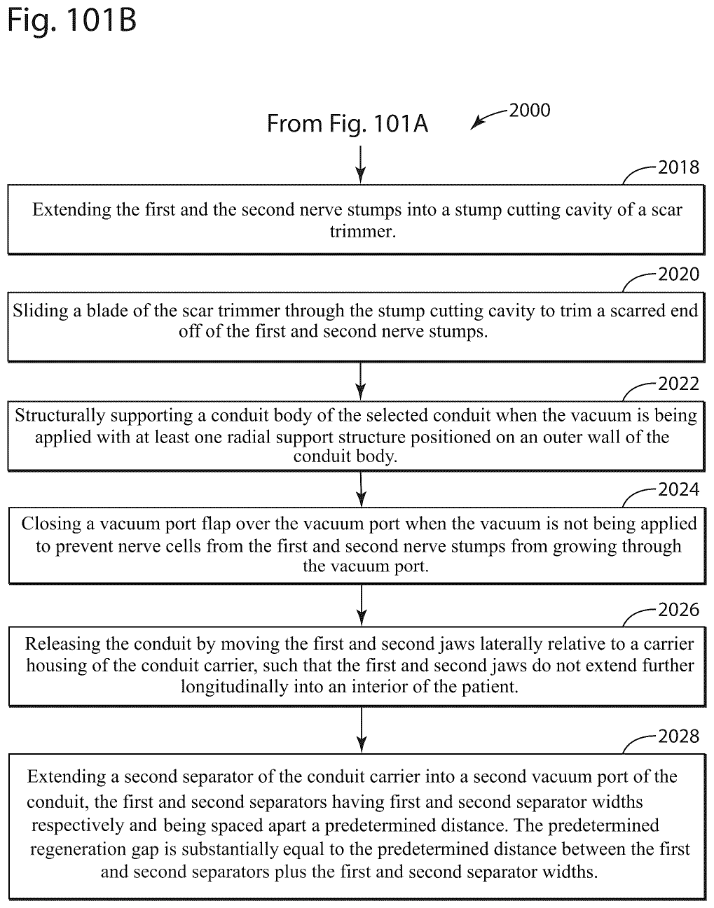

Referring to FIG. 101A, a flow diagram is depicted of a method 2000 of performing a nerve repair procedure on a patient, in accordance with aspects of the present disclosure. The method begins at 2002, wherein a first nerve stump 410 is fitted into a U-shaped recess 380 of a nerve sizing instrument 350 to determine a diameter of the first nerve stump 410.

In the case where the U-shaped recess 380 has a depth and a width that is equal to a predetermined maximum diameter of a nerve stump 410 that can fit into the recess 380, the diameter of the first nerve stump 410 may be determined by hand. More specifically, the diameter of the nerve stump 410 may be determined by feeling that the top of the nerve stump 410 is level with the top of the U-shaped recess 380.

A diameter of a second nerve stump 410 which is going to be coapted to the first nerve stump may also be determined in the same manner. However, this step may not be necessary since the two nerve stumps 410 are more than likely substantially the same diameter.

At 2004 of the method, a conduit 100, 170, 200 and a conduit carrier 660, 930, 1500, 1650 1700 are selected based on the determined diameter of the first nerve stump 410.

At 2006, the selected conduit 100, 170, 200 is releasably engaged with the selected conduit carrier 660, 1500, 1650, 1700. Releasably engaging the selected conduit 100, 170, 200 with the selected conduit carrier 660, 1500, 1700 may be done during the nerve repair procedure in, for example, an operating room. Alternatively, the releasably engaging may be done prior to the commencement of the nerve repair procedure at a remote location, such as, for example, a medical equipment factory or distribution facility.

At 2008, a first separator 710, 800 of the conduit carrier 660, 1500 is extended into a vacuum port 110 of the conduit 100. Extending the first separator 710, 800 into the vacuum port 110 of the conduit 100 may be done during the nerve repair procedure in, for example, an operating room. Alternatively, the extending may be done prior to the commencement of the nerve repair procedure at a remote location, such as, for example, a medical equipment factory or distribution facility.

At 2010, a vacuum channel 775 of the conduit carrier 660, 1500 is connected to the vacuum port 110 of the conduit 100. Connecting the vacuum channel 775 to the vacuum port 110 may be done during the nerve repair procedure in, for example, an operating room. Alternatively, the connecting may be done prior to the commencement of the nerve repair procedure at a remote location, such as, for example, a medical equipment factory or distribution facility.

At 2012, a vacuum 850 is pulled, via the vacuum channel 775, on an interior of the conduit 100.

At 2014, the first and the second nerve stumps 410 are inserted into first and second open ends 150 respectively of the conduit 100 while pulling the vacuum 850.

At 2016, the first separator 800 is utilized to provide a predetermined regeneration gap 895 between the first and second nerve stumps 410. The first separator may have a separator width of between 0.1 and 5 millimeters and the predetermined regeneration gap 895 may be substantially equal to the separator width.

Referring to FIG. 101B, a continuation of the flow diagram of method 2000 is depicted, in accordance with aspects of the present disclosure, wherein some of the steps depicted may or may not be required or utilized.

The method may continue at 2018, wherein the first and the second nerve stumps 410 are extended into a stump cutting cavity 480 of a scar trimmer 450.

At 2020 of method 2000, a blade 500 of the scar trimmer 450 may be slid through the stump cutting cavity 480 to trim a scarred end off of the first and second nerve stumps 410. The trimming of scar tissue off of the nerve stumps 410 will occur before the nerve stumps 410 are inserted into the conduit 100.

At 2022, a conduit body of the selected conduit 100, 200 may be structurally supported when the vacuum is being applied with at least one radial support structure 130, 220 positioned on an outer wall 140 of the conduit body.

The at least one radial support structure may include at least one retaining tab 130. The at least one retaining tab 130 may be releasably engaged with first and second jaws 1520 of the conduit carrier 1500 to structurally support the conduit body when the vacuum 850 is being applied.

At 2024, a vacuum port flap 120 may be closed over the vacuum port 110 when the vacuum is not being applied to prevent nerve cells from the first and second nerve stumps 410 from growing through the vacuum port 110.

At 2026, the conduit 100 may be released by moving the first and second jaws 1520 laterally relative to a carrier housing 1510 of the conduit carrier 1500, such that the first and second jaws 1520 do not extend further longitudinally into an interior of the patient.

At 2028, a second separator 800 of the conduit carrier 1500 may be extended into a second vacuum port 110 of the conduit 1800. The first and second separators 800 have first and second separator widths respectively and are spaced apart a predetermined distance. The predetermined regeneration gap 895 is substantially equal to the predetermined distance between the first and second separators 800 plus the first and second separator widths.

It is contemplated that a kit for performing a nerve repair procedure will be utilized during the surgical procedure. The kit may contain the following elements: a plurality of different sized conduits, at least one conduit carrier, a separator, a nerve sizing instrument, a scar trimmer, and a vacuum source for facilitating the nerve repair procedure.

It should be appreciated that all combinations of the foregoing concepts and additional concepts discussed in greater detail herein (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

As may be recognized by those of ordinary skill in the art based on the teachings herein, numerous changes and modifications may be made to the above-described and other embodiments of the present disclosure without departing from the scope of the disclosure. The components of the implants as disclosed in the specification, including the accompanying abstract and drawings, may be replaced by alternative component(s) or feature(s), such as those disclosed in another embodiment, which serve the same, equivalent or similar purpose as known by those skilled in the art to achieve the same, equivalent or similar results by such alternative component(s) or feature(s) to provide a similar function for the intended purpose. Accordingly, this detailed description of the currently-preferred embodiments is to be taken in an illustrative, as opposed to limiting of the disclosure.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as “has”, and “having”), “include” (and any form of include, such as “includes” and “including”), and “contain” (and any form of contain, such as “contains” and “containing”) are open-ended linking verbs. As a result, a method or device that “comprises,” “has,” “includes,” or “contains” one or more steps or elements possesses those one or more steps or elements, but is not limited to possessing only those one or more steps or elements. Likewise, a step of a method or an element of a device that “comprises,” “has,” “includes,” or “contains” one or more features possesses those one or more features, but is not limited to possessing only those one or more features. Furthermore, a device or structure that is configured in a certain way is configured in at least that way, but may also be configured in ways that are not listed.

The disclosure has been described with reference to the preferred embodiments. It will be understood that the architectural and operational embodiments described herein are exemplary of a plurality of possible arrangements to provide the same general features, characteristics, and general system operation. Modifications and alterations will occur to others upon a reading and understanding of the preceding detailed description. It is intended that the disclosure be construed as including all such modifications and alterations. Accordingly, it is intended that the disclosure is not limited to the described examples, but that it has the full scope defined by the language of the following claims.

What is claimed is:

1. A conduit comprising:
a conduit body including opposing first and second open end portions and a hollow conduit interior extending therebetween, the conduit interior configured to receive a first nerve stump through the first open end portion and a second nerve stump through the second open end portion during a nerve repair procedure on a patient;
a vacuum port extending through an outer wall of the conduit body, the vacuum port configured to connect to a vacuum source associated with a conduit carrier to pull a vacuum on the conduit interior while inserting the nerve stumps into the first and second open end portions; and
at least one radial support structure positioned on the outer wall of the conduit body, the at least one radial support structure configured to enable jaws of the conduit carrier to engage therewith, wherein the engagement of the jaws of the conduit carrier with the at least one radial support structure provides structural support for the conduit body when the vacuum is being applied.

2. The conduit of claim 1, comprising:
at least one vacuum port flap configured to close over the vacuum port when the vacuum is not being applied, wherein when the at least one vacuum port flap is closed over the vacuum port, the at least one vacuum port flap prevents nerve cells from the first or second nerve stumps from growing through the vacuum port.

3. The conduit of claim 1, wherein the vacuum port comprises a first and a second vacuum port disposed on the outer wall of conduit body.

4. The conduit of claim 1, wherein the at least one radial support structure comprises at least one retaining tab positioned on the outer wall of the conduit body, the at least one retaining tab configured to enable the jaws of the conduit carrier to engage therewith.

5. The conduit of claim 4, comprising:
the at least one retaining tab comprises:
a first set of retaining tabs being positioned proximate the first open end portion, and
a second set of retaining tabs being positioned proximate the second open end portion; and
wherein the vacuum port is positioned centrally on the conduit body between the first and second sets of retaining tabs.

6. The conduit of claim 5, wherein:
the first set of retaining tabs comprise three tabs spaced apart between 60 and 180 degrees on the outer wall of the conduit body; and
the second set of retaining tabs comprise three tabs spaced apart between 60 and 180 degrees on the outer wall of the conduit body.

7. The conduit of claim 4, comprising:
at least one retaining tab opening disposed on the outer wall adjacent the at least one retaining tab;
wherein the at least one retaining tab is foldable into the at least one retaining tab opening.

8. The conduit of claim 7, comprising:
the at least one retaining tab comprising a plurality of retaining tabs and the at least one retaining tab opening comprising a plurality of retaining tab openings;
wherein each retaining tab of the plurality of retaining tabs is foldable into an associated adjacent retaining tab opening of the plurality of retaining tab openings.

9. The conduit of claim 4, wherein the at least one retaining tab comprises:
a continuous support tab extending longitudinally along a length of the conduit body from the first open end portion to the second open end portion.

10. The conduit of claim 4, wherein the at least one retaining tab comprises a linked tab, the linked tab comprising:
a first and a second radial tab segment extending radially outward from the outer wall; and
a linking tab segment integrally connected to the first and second radial tab segments and extending longitudinally therebetween;
wherein the linked tab and outer wall define an open capture recess therebetween.

11. The conduit of claim 4, wherein the at least one retaining tab comprises a dovetail tab, the dovetail tab comprising:
a lower tab end attached to the outer wall, the lower tab end having a first width; and
an upper tab end having a second width;
wherein the second width is larger than the first width.

12. The conduit of claim 4, wherein the at least one retaining tab comprises a stepped tab, the stepped tab comprising:
a lower tab segment attached to the outer wall, the lower tab segment having a first width; and
an upper tab segment disposed over the lower tab segment, the upper tab segment having a second width;
wherein the second width is larger than the first width.

13. The conduit of claim 1, wherein the conduit body comprises a spiral wall, the spiral wall comprising:
a longitudinally extending first wall end portion and a longitudinally extending second wall end portion;
wherein the first wall end portion overlaps and abuts against the second wall end portion to form the hollow conduit interior.

14. The conduit of claim 1, wherein the conduit comprises a stepped diameter conduit, wherein the stepped diameter conduit comprises:
the first open end portion having a first internal diameter;
the second open end portion having a second internal diameter; and
a central section of the conduit body disposed between the first and second end portions, the central section having a third internal diameter;
wherein the first and second internal diameters are smaller than the third internal diameter.

15. The stepped diameter conduit of claim 14, comprising:
a plurality of first elasticity tuning openings disposed in the first open end portion, the first elasticity tuning openings configured to provide a predetermined elasticity to the first open end portion; and
a plurality of second elasticity tuning openings disposed in the second open end portion, the second elasticity tuning openings configured to provide a predetermined elasticity to the second open end portion.

16. The conduit of claim 1, wherein the conduit comprises a rolled end conduit, wherein the at least one radial support structure comprises:
the first open end portion being configured to be rolled back upon itself from a first extended position to a first rolled back position, wherein in the first rolled back position the first open end portion:
structurally supports the conduit body when the vacuum is being applied, and
forms a first nerve entry region configured to receive the first nerve stump; and
the second open end portion being configured to be rolled back upon itself from a second extended position to a second rolled back position, wherein in the second rolled back position the second open end portion:
structurally supports the conduit body when the vacuum is being applied, and
forms a second nerve entry region configured to receive the second nerve stump.

17. The rolled end conduit of claim 16, comprising:
a first unrolling tab detachably connected to a first distal end of the first open end portion, the first unrolling tab configured to provide leverage to roll back the first open end portion from the first extended position to the first rolled back position; and
a second unrolling tab detachably connected to a second distal end of the second open end portion, the second unrolling tab configured to provide leverage to roll back the second open end portion from the second extended position to the second rolled back position.

18. The rolled end conduit of claim 16, comprising:
a plurality of first elasticity tuning openings disposed in the first open end portion, the first elasticity tuning openings configured to provide a predetermined elasticity to the first open end portion; and
a plurality of second elasticity tuning openings disposed in the second open end portion, the second elasticity tuning openings configured to provide a predetermined elasticity to the second open end portion.

19. A conduit carrier comprising:
a carrier housing comprising first and second contact ends and a vacuum channel extending therebetween, the vacuum channel configured to connect to a vacuum source through the first contact end;
an engagement mechanism connected to the second contact end, the engagement mechanism configured to releasably engage a conduit during a nerve repair procedure on a patient; and
a separator configured to extend from the second contact end;
wherein, when the engagement mechanism is engaged with the conduit during the nerve repair procedure:
the vacuum channel is connected through the second contact end to a vacuum port of the conduit to enable the vacuum source to pull a vacuum on an interior of the conduit, and
the separator extends into the interior of the conduit through the vacuum port to provide a predetermined regeneration gap between a first and a second nerve stump inserted into the conduit.

20. A nerve sizing instrument for sizing a nerve stump during a nerve repair procedure on a patient, the nerve sizing instrument comprising a plurality of U-shaped recesses, wherein:
respective recesses of the plurality of U-shaped recesses are configured to receive therein a nerve stump having up to a predetermined nerve stump maximum diameter that is different from any other nerve stump maximum diameter associated with any other respective recess.

21. A scar trimmer for trimming a nerve stump during a nerve repair procedure on a patient, the scar trimmer comprising:
a longitudinally extending housing assembly having a forward end and a rearward end, the forward end having a forward boss disposed thereon, the forward boss having a stump cutting cavity disposed therein, the stump cutting cavity configured to receive a nerve stump extended therethrough;
an internal slider disposed within an interior of the housing assembly, the internal slider movable between a forward position and a rearward position relative to the housing assembly; and
a blade slidably captured by the internal slider;
wherein, when a nerve stump is extended through the stump cutting cavity and the internal slider is moved between the rearward position and the forward position, the blade is slide through the stump cutting cavity to trim a scarred end of the nerve stump off.

22. A kit for performing a nerve repair procedure, the kit comprising:
a plurality of conduits,
at least one conduit carrier,
at least one nerve sizing instrument,
at least one scar trimmer,
at least one separator, and
a vacuum source for facilitating the nerve repair procedure.

23. A method of performing a nerve repair procedure on a patient, the method comprising:
selecting a conduit and a conduit carrier based on a determined diameter of a first nerve stump;
pulling a vacuum, via a vacuum channel of the conduit carrier, on an interior of the conduit;
inserting the first and a second nerve stump into first and second open ends respectively of the conduit while pulling the vacuum; and
utilizing a first separator of the conduit carrier to provide a predetermined regeneration gap between the first and second nerve stumps.
